(19) United States
(12) Patent Application Publication
(10) Pub. No.: US 2023/0321144 A1
(43) Pub. Date: Oct. 12, 2023

(54) MODIFIED IMMUNE CELLS FOR FIBROSIS AND INFLAMMATION

(71) Applicant: Carisma Therapeutics Inc., Philadelphia, PA (US)

(72) Inventors: Aditya Dandekar, Philadelphia, PA (US); Michael Klichinsky, New Hope, PA (US)

(21) Appl. No.: 18/023,060

(22) PCT Filed: Aug. 27, 2021
(86) PCT No.: PCT/US21/47889

§ 371 Date: Feb. 24, 2023

Related U.S. Application Data
(60) Provisional application No. 63/072,046, filed on Aug. 28, 2020.

(51) Int. Cl. A61K 35/15; A61P 29/00; A61K 38/20; C12N 15/86

(52) U.S. Cl. CPC A61K 35/15; C12N 15/86; A61K 38/2066; A61P 29/00

(57) ABSTRACT

The present disclosure pertains to immune cells comprising exogenous fibrolytic agents and/or exogenous anti-inflammatory agents and methods of using immune cells comprising exogenous fibrolytic agents and/or exogenous anti-inflammatory agents.

### CROSS REFERENCE TO RELATED APPLICATIONS

This application claims priority to and the benefit of U.S. Provisional Patent Application No. 63/072,046, filed Aug. 28, 2020; the contents of which are incorporated herein by reference in its entirety.

### BACKGROUND

Fibrosis is a serious health problem characterized by the development of excess fibrous connective tissue due at least in part to reparative and/or reactive processes, such as in response to an injury. Fibrosis can occur in various organs including the lung, liver, heart, kidney, pancreas, skin, and brain. Currently available therapies for fibrotic diseases, disorders, and conditions have limited efficacy.

Inflammatory diseases are a significant cause of morbidity and mortality in humans. There are various side effects associated with currently available treatments for inflammation, such as adrenal suppression, weakening of bones, muscle wasting, peptic ulcers, hypokalemia, and immune system suppression.

Therefore, a need exists for the development of new therapeutic modalities optimized to reduce and treat fibrosis and inflammation.

### SUMMARY OF THE INVENTION

The present disclosure pertains to immune cells comprising fibrolytic agents and/or exogenous anti-inflammatory agents and methods of using immune cells comprising fibrolytic agents and/or exogenous anti-inflammatory agents. In part, the present disclosure encompasses the recognition that the administration of specifically modified immune cells is surprisingly effective in treating one or more symptoms of fibrotic and/or inflammatory diseases.

In one aspect, the disclosure provides modified immune cells comprising one or more nucleic acid sequences encoding: (i) at least one exogenous fibrolytic agent, and/or (ii) at least one exogenous anti-inflammatory agent.

In some embodiments, at least one exogenous fibrolytic agent comprises a matrix metallopeptidase (MMP), or TWEAK polypeptide. In some embodiments, a MMP comprises or is one or more of MMP-1, MMP-2, MMP-3, MMP-7, MMP-8, MMP-9, MMP-12, MMP-13, MMP-14, MMP-15, MMP-16, MMP-19, MMP-24, and/or TIMP-1.

In some embodiments, at least one exogenous anti-inflammatory agent comprises or is a cytokine, a chemokine, or a pentraxin. In some embodiments, a cytokine comprises or is IL10, IL-4, IL-13, and/or TGF-beta. In some embodiments, a chemokine comprises or is CX3CL. In some embodiments, a pentraxin comprises or is Pentraxin-2.

In some embodiments, at least one exogenous fibrolytic agent and/or the at least one exogenous anti-inflammatory agent are tethered to the immune cell or secreted from the immune cell.

In some embodiments, one or more nucleic acid sequences comprise one or more liver specific promoters or cirrhosis specific promoters. In some embodiments, one or more nucleic acid sequences comprise a CX3CR1 promoter, an insulin-like growth factor 1 (IGF1), or a CD11B promoter.

In some embodiments, a modified immune cell comprises a macrophage, monocyte, or dendritic cell. In some embodiments, a macrophage comprises or is a polarized macrophage. In some embodiments, a polarized macrophage comprises or is an M0, M1, or M2 macrophage. In some embodiments, a M2 macrophage comprises or is a M2A, M2B, M2C, or M2D macrophage. In some embodiments, a macrophage is derived from a monocyte or a precursor immune cell. In some embodiments, a precursor immune cell comprises or is a hematopoietic stem cell, myeloid progenitor, myeloblast, monoblast, promonocyte, or an intermediate thereof. In some embodiments, a macrophage is a G-MCSF derived macrophage or an M-CSF derived macrophage.

In some embodiments, a modified immune cell described herein (e.g., comprising at least one exogenous anti-inflammatory agent) exhibits an M2 phenotype relative to a second modified immune cell that does not comprise one or more nucleic acid sequences encoding at least one exogenous anti-inflammatory agent. In some embodiments, a modified immune cell described herein maintains an M2 phenotype for at least 7 days. In some embodiments, a modified immune cell described herein (e.g., comprising at least one exogenous anti-inflammatory agent) exhibits increased expression of one or more markers of a M2 phenotype relative to a second modified immune cell that does not comprise one or more nucleic acid sequences encoding at least one exogenous anti-inflammatory agent. In some embodiments, one or more markers of M2 phenotype comprise or are one, two, or three of CD163, CD206, or CD209. In some embodiments, secretion of an anti-inflammatory agent (e.g., IL10) from a modified immune cell described herein converts one or more unmodified immune cells into an M2 phenotype.

In another aspect, the disclosure provides pharmaceutical compositions comprising a modified immune cell of any aspect or embodiment described herein. In some embodiments, a pharmaceutical composition comprises a pharmaceutically acceptable carrier.

In another aspect, the disclosure provides nucleic acid constructs comprising one or more nucleic acid sequences encoding least one exogenous fibrolytic agent of any aspect or embodiment described herein and/or at least one exogenous anti-inflammatory agent of any aspect or embodiment described herein.

In another aspect, the disclosure provides pharmaceutical compositions comprising a nucleic acid construct of any aspect or embodiment described herein. In some embodiments, a pharmaceutical composition comprises a pharmaceutically acceptable carrier.

In another aspect, the disclosure provides methods of treating or preventing fibrosis or inflammation in a subject, comprising delivering to a subject a therapeutically effective amount of a pharmaceutical composition of any aspect or embodiment described herein.

In some embodiments, fibrosis comprises or is a liver, lung, heart, vasculature, kidney, pancreas, skin, gastrointestinal, bone marrow, hematopoietic tissue, nervous system, and/or eye fibrotic disease, disorder, or condition. In some embodiments, a liver fibrotic disease, disorder, or condition comprises a fatty liver disease, disorder, or condition. In some embodiments, a fatty liver disease, disorder, or condition comprises non-alcoholic fatty liver disease (NAFL) or alcoholic liver disease. In some embodiments, NAFL comprises non-alcoholic fatty liver disease (NAFLD) or non-alcoholic steatohepatitis (NASH). In some embodiments, alcoholic liver disease comprises alcoholic fatty liver disease (AFLD) or alcoholic steatohepatitis (ASH). In some embodiments, a subject has one or more of cirrhosis, liver damage, hepatocarcinoma, steatosis, an increased risk of liver failure, an increased risk of death, and/or Hepatitis C infection (HCV).

In some embodiments, inflammation comprises or is a liver, gastrointestinal tract, lung, skin, cardiovascular system, nervous system, kidney, pancreas, joint, eye, and/or an endocrine system inflammatory disease, disorder, or condition.

In some embodiments, methods described herein reduce activation of hepatic stellate cells. In some embodiments, methods described herein improve liver regeneration and/or liver resolution. In some embodiments, methods described herein balance pro-fibrotic and anti-fibrotic macrophage populations.

In another aspect, the disclosure provides methods of modifying an immune cell, comprising delivering to an immune cell a nucleic acid construct comprising one or more nucleic acid sequences encoding at least one exogenous anti-fibrotic agent of any aspect or embodiment described herein and/or at least one exogenous anti-inflammatory agent of any aspect or embodiment described herein.

In some embodiments, delivering comprises electroporation or transfection with mRNA, DNA, or chemically modified mRNA. In some embodiments, delivering comprises transduction with an adeno-associated viral (AAV) vector, an adenoviral vector, or a retroviral vector. In some embodiments, a retroviral vector comprises a lentiviral vector or a gammaretroviral vector. In some embodiments, delivering comprises transduction with a viral vector (e.g., a lentiviral vector) and at least one Vpx protein. In some embodiments, an immune cell described herein is one or both of electroporated or transfected with at least one Vpx mRNA prior to and/or concurrently with, transfection with the viral vector. In some embodiments, a lentiviral vector is packaged with a Vpx protein. In some embodiments, an adenoviral vector comprises an Ad2 vector or an Ad5 vector. In some embodiments, an Ad5 vector comprises an Ad5f35 adenoviral vector. In some embodiments, delivery comprises transposon-based delivery or CRISPR-based targeted integration (e.g., CRISPR/Cas systems comprising one or more of Cas9, Cas12a, or C2c2).

### BRIEF DESCRIPTION OF THE DRAWING

The drawings are for illustration purposes only, not for limitation.

FIG. 1 is a series of graphs showing viability of human macrophages at 3 Days after electroporation with 30 nM, 100 nM and 300 nM of IL10 mRNA or mCherry mRNA.

FIGS. 2A-2B are a series of graphs showing IL10 levels in supernatant of human macrophages from donor 1 (FIG. 2A) and donor 2 (FIG. 2B) at Day 3, Day 5, and Day 7 after electroporation with 30 nM, 100 nM and 300 nM of IL10 mRNA or mCherry mRNA.

FIGS. 3A-3B are a series of graphs showing MFI of M2 markers (CD163 and CD206) and M1 markers (CD80 and CD86) of human macrophages from donor 1 (FIG. 3A) and donor 2 (FIG. 3B) at Day 3 after electroporation with 30 nM, 100 nM and 300 nM of IL10 mRNA or mCherry mRNA.

FIGS. 4A-4B are a series of graphs showing MFI of M2 markers (CD163 and CD206) of human macrophages from donor 1 (FIG. 4A) and donor 2 (FIG. 4B) at Day 7 after electroporation with 30 nM, 100 nM and 300 nM of IL10 mRNA or mCherry mRNA.

FIGS. 5A-5B are a series of graphs showing MFI of M2 markers (CD163 and CD206) of human macrophages from donor 1 (FIG. 5A) and donor 2 (FIG. 5B) after stimulation with IL10 secreted from engineered macrophages electroporated with 30 nM, 100 nM and 300 nM of IL10 mRNA or mCherry mRNA.

FIGS. 6A-6B are a series of graphs showing IL10 levels in supernatant of human macrophages from donor 1 (FIG. 6A) and donor 2 (FIG. 6B) at Day 3, Day 5, and Day 7 after transduction with VPX lentivirus (VPX-LV) encoding IL10 or VPX-LV encoding GFP.

FIGS. 7A-7B are a series of graphs showing MFI of M2 markers (CD163 and CD206) and M1 markers (CD80 and CD86) of human macrophages at Day 3 (FIG. 7A) and Day 7 (FIG. 7B) after transduction with VPX-LV encoding IL10 or VPX-LV encoding GFP.

FIGS. 8A-8B are a series of graphs showing MFI of M2 markers (CD163 and CD206) and M1 markers (CD80 and CD86) of human macrophages from donor 1 (FIG. 8A) and donor 2 (FIG. 8B) after stimulation with IL10 secreted from engineered macrophages transduced with VPX-LV encoding IL10 or VPX-LV encoding GFP.

FIGS. 9A-9B are a series of graphs showing MMP13 levels in supernatant of human macrophages at Day 1, Day 3, and Day 5 after electroporation with MMP13 mRNA or f-luc mRNA (FIG. 9A) and at Day 5, Day 7, and Day 10 after transduction with MMP13 VPX-LV or VPX-LV encoding GFP (FIG. 9B).

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Activation: As used herein, the term “activation” refers to the state of a cell, for example a monocyte, macrophage, or dendritic cell that has been sufficiently stimulated to induce detectable cellular proliferation or has been stimulated to exert its effector function. Activation can also be associated with induced cytokine production, cytokine secretion, phagocytosis, cell signaling (e.g., gene expression changes), target cell killing, metabolic changes, production of inflammatory mediators, proliferation, epigenetic reprogramming, phenotypic switching of macrophages (e.g., M1 polarization), suppression of pro-tumor or M2 macrophages, phenotypic switching of pro-tumor or M2 macrophages, and/or antigen processing and presentation.

Activated monocytes/macrophages/dendritic cells: As used herein, the term “activated monocytes/macrophages/dendritic cells” refers to, among other things, monocyte/macrophage/dendritic cells that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, production of inflammatory mediators, proliferation, epigenetic reprogramming, phenotypic switching of macrophages (e.g., M1 polarization), suppression of pro-tumor or M2 macrophages, phenotypic switching of pro-tumor or M2 macrophages, and other functions.

Agent: As used herein, the term “agent” (or “biological agent” or “therapeutic agent”), refers to a molecule that may be expressed, released, secreted or delivered to a target by a modified immune cell described herein. An agent includes, but is not limited to, a nucleic acid, an antibiotic, an antibody or fragments thereof, an antibody agent or fragments thereof, a growth factor, a cytokine, an enzyme, a protein, a peptide, a fusion protein, a synthetic molecule, an organic molecule (e.g., a small molecule), a carbohydrate, a lipid, a hormone, a microsome, a derivative or a variation thereof, a formulation or composition including one or more thereof, and any combinations thereof. An agent may bind any cell moiety, such as a receptor, an antigenic determinant, or other binding site present on a target or target cell. An agent may diffuse or be transported into a cell, where it may act intracellularly. For Example, an agent may be an exogenous fibrolytic agent as described herein. An agent may also an exogenous anti-inflammatory agent as described herein.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with methods and compositions described herein is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antibody fragment: As used herein, the term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to a graft (e.g., a population of cells) derived from a different animal of the same species.

Xenogenic: As used herein, the term “xenogeneic” refers to a graft (e.g., a population of cells) derived from an animal of a different species.

Conservative sequence modifications: As used herein, the term “conservative sequence modifications” refers to amino acid modifications that do not significantly affect or alter at least one property or characteristic of a particular protein, for example, the binding characteristics of an antibody, containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Effective amount: As used herein, “effective amount” and “therapeutically effective amount” are interchangeable, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

Effector function: As used herein, “effector function” or “effector activity” refers to a specific activity carried out by an immune cell in response to stimulation of the immune cell. For example, an effector function of macrophages to engulf and digest cellular debris, foreign substances, microbes, cancer cells and other unhealthy cells by phagocytosis.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of monocytes/macrophages. In one embodiment, monocytes, macrophages, or dendritic cells that are expanded ex vivo increase in number relative to the number originally present in the culture. In another embodiment, monocytes, macrophages, or dendritic cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (e.g., Ad5f35) that incorporate the recombinant polynucleotide.

Fibrosis: As used herein, the term “fibrosis” refers to formation of fibrous tissue as a reparative and/or reactive process, rather than as a normal constituent of a cell, tissue, or organ. Fibrosis is characterized by fibroblast accumulation and collagen deposition in excess of normal deposition in any particular tissue. Fibrotic diseases, disorders, and conditions include, but are not limited to, fibrosis arising from wound healing, systemic and local scleroderma, atherosclerosis, restenosis, pulmonary inflammation, idiopathic pulmonary fibrosis, interstitial lung disease, liver cirrhosis, fibrosis as a result of chronic hepatitis B or C infection, kidney disease (e.g., glomerulonephritis), heart disease resulting from scar tissue, keloids and hypertrophic scars, and eye diseases (e.g., macular degeneration, retinal retinopathy, and vitreal retinopathy). Fibrotic diseases, disorders, and conditions can be hepatic-related, such as diseases, disorders, and conditions resulting from accumulation of cholesterol and/or triglycerides within hepatocytes, which may result in a pro-inflammatory response that leads to liver fibrosis and/or cirrhosis. Hepatic disorders having a fibrotic component include, but are not limited to, non-alcoholic fatty liver disease (NAFL) (e.g., non-alcoholic fatty liver disease (NAFLD) or non-alcoholic steatohepatitis (NASH)) or alcoholic liver disease (e.g., alcoholic fatty liver disease (AFLD) or alcoholic steatohepatitis (ASH)). Fibrotic diseases, disorders, and conditions can include mechanical trauma, biliary obstruction, autoimmune hepatitis, iron overload, Hepatitis B infection (HBV), and/or Hepatitis C infection (HCV).

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Inflammation: As used herein, the term “inflammation” refers to a cellular, physiological, and/or immunological response to noxious stimuli including, but not limited to, injuries, immunologic reactions, infections, altered endogenous substances, defective endogenous pathways, and/or foreign substances. Inflammation can be systemic or local. Inflammation can be acute or chronic. Exemplary inflammatory conditions, diseases, and disorders include, but are not limited, to a liver, a gastrointestinal tract inflammatory, a lung, a skin a cardiovascular system, a nervous system, a kidney, a pancreas, a joint, an eye, and/or an endocrine system inflammatory condition, disease, or disorder. An inflammatory disease, disorder, or condition can be associated with a toxin, an insult (e.g., an environmental hazard (e.g., asbestos, coal dust, and/or polycyclic aromatic hydrocarbons) or cigarette smoking), and/or a medical treatment (e.g., surgery). An inflammatory disease, disorder, or condition can be or include an autoimmune disorder.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Lentivirus: As used herein, the term “lentivirus” refers to a genus of the Retroviridae family. Lentiviruses are unique among the retroviruses in being able to infect non-dividing cells; they can deliver a significant amount of genetic information into the DNA of a host cell, so they are one of the most efficient methods of a gene delivery vector. HIV, SIV, and FIV are all examples of lentiviruses. Vectors derived from lentiviruses offer the means to achieve significant levels of gene transfer in vivo.

Modified: As used herein, the term “modified” refers to a changed state or structure of a molecule or cell of the disclosure. Molecules may be modified in many ways, including chemically, structurally, and functionally. Cells may be modified through the introduction of nucleic acids.

Modulating: As used herein the term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Operably linked: As used herein, the term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially purified: As used herein, the term “substantially purified”, for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition (e.g., a disease, disorder, and/or condition associated with fibrosis and/or inflammation). In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

### Immune Cells

The present disclosure, among other things, provides modified immune cells (e.g., macrophages, monocytes, or dendritic cells) comprising one or more nucleic acid sequences encoding at least one exogenous fibrolytic agent (e.g., a polypeptide described herein) and/or at least one exogenous anti-inflammatory agent (e.g., a polypeptide described herein). Modified immune cells (e.g., macrophages, monocytes, or dendritic cells) can comprises or express at least one exogenous fibrolytic agent (e.g., a polypeptide described herein) and/or at least one exogenous anti-inflammatory agent (e.g., a polypeptide described herein).

As used herein, the term “immune cell,” refers to a cell that is involved in an immune response, e.g., promotion of an immune response. Examples of immune cells include, but are not limited to, macrophages, monocytes, dendritic cells, neutrophils, eosinophils, mast cells, platelets, large granular lymphocytes, Langerhans' cells, natural killer (NK) cells, T-lymphocytes, or B-lymphocytes. A source of immune cells (e.g., macrophages, monocytes, or dendritic cells) can be obtained from a subject.

In some embodiments, provided methods and/or compositions include a population of immune cells. In some embodiments, a population of immune cells as described herein comprises monocytes, macrophages, dendritic cells, and/or precursors thereof. In some embodiments, a population of immune cells comprises a purified population of monocytes, macrophages, or dendritic cells, or a cell line.

In some embodiments, an immune cell is activated, e.g., an immune cell exhibits increased cytokine production, chemokine production, phagocytosis, cell signaling, target cell killing, and/or antigen presentation, e.g., relative to an inactive cell. In some embodiments, an activated immune cell exhibits changes in gene expression, e.g., an induction of pro-inflammatory gene expression (e.g., one, two, three, four, five, six, or seven of TNF, IL-12, IFN, GM-CSF, G-CSF, M-CSF, or IL-1), e.g., relative to an inactive cell. In certain embodiments, activated immune cells are undergoing cell division.

In some embodiments, immune cells (e.g., macrophages, monocytes, or dendritic cells) are obtained (e.g., isolated) from a subject. Immune cells may be autologous or sourced from allogeneic or universal donors. Cells can be obtained from a number of sources including peripheral blood mononuclear cells, bone marrow, lymph node tissue, spleen tissue, umbilical cord, tumors, and/or induced pluripotent stem cells, such as embryonic stem cells (ESCs). In certain embodiments, cells can be obtained from a unit of blood collected from a subject using any number of separation techniques known to a skilled artisan, such as Ficoll separation. In some embodiments, cells from circulating blood of a subject are obtained by apheresis or leukapheresis. Cells collected by apheresis may be washed to remove a plasma fraction and resuspended in a variety of buffers (e.g., phosphate buffered saline (PBS)) or culture media. In some embodiments, enrichment of immune cells (e.g. monocytes) comprises plastic adherence. In some embodiments, following enrichment, differentiation of immune cells (e.g. monocytes) comprises stimulation with GM-CSF. In some embodiments, a composition comprising blood cells (e.g., monocytes, lymphocytes, platelets, plasma, and/or red blood cells), such as a leukapheresis composition (e.g., a leukopak) is used for enrichment. In some embodiments, a leukapheresis composition (e.g., a leukopak) comprises a sample from a healthy human donor. In certain embodiments, apheresis of immune cells (e.g. monocytes) is followed by mobilization with GM-CSF. In certain embodiments, selection of immune cells (e,g, monocytes) comprises CD14 positive selection using microbeads (e.g., MACS® MicroBeads on a CliniMACS Prodigy device).

In some embodiments, an immune cell precursor (e.g., precursors to macrophages, monocytes, or dendritic cells) is used in compositions and methods described herein. Immune cell precursors may be differentiated in vivo or ex vivo into immune cells. Non-limiting examples of precursor immune cells include hematopoietic stem cells, myeloid progenitors, myeloblasts, monoblasts, promonocytes, or intermediates thereof. For example, induced pluripotent stem cells may be used to generate monocytes, macrophages, and/or dendritic cells. Induced pluripotent stem cells (iPSCs) may be derived from normal human tissue, such as peripheral blood, fibroblasts, skin, keratinocytes, or renal epithelial cells. Autologous, allogeneic, or universal donor iPSCs could be differentiated toward a myeloid lineage (e.g., a monocyte, macrophage, dendritic cell, or precursor thereof).

Immune cells (e.g., macrophages, monocytes, or dendritic cells) as described herein can be isolated from peripheral blood, for example, by lysing red blood cells and depleting lymphocytes and red blood cells, such as by centrifugation through a PERCOLL™ gradient. Alternatively, immune cells can be isolated from umbilical cord tissue. A specific subpopulation of immune cells can be further isolated by positive or negative selection techniques. In some embodiments, immune cells can be depleted of cells expressing certain antigens, including, but not limited to, CD34, CD3, CD4, CD8, CD56, CD66b, CD19, or CD20. In some embodiments, enrichment of an immune cell population, for example, by negative selection can be accomplished using a combination of antibodies directed to surface markers unique to the negatively selected cells. By way of non-limiting example, cell selection can also comprise negative magnetic immunoadherence or flow cytometry that uses a cocktail of monoclonal antibodies directed to cell surface markers present on negatively selected cells.

During isolation of a desired population of immune cells (e.g., macrophages, monocytes, or dendritic cells) as described herein by positive or negative selection, immune cell concentration and surface (e.g., particles, such as beads) can be varied. It may be desirable to significantly decrease volume in which beads and cells are mixed together to ensure maximum contact area of cells and beads.

### Macrophages

The present disclosure, among other things, provides macrophages comprising one or more nucleic acid sequences encoding at least one exogenous fibrolytic agent (e.g., a polypeptide described herein) and/or at least one exogenous anti-inflammatory agent (e.g., a polypeptide described herein). Macrophages are immune cells specialized for detection, phagocytosis, and destruction of target cells, such as pathogens or tumor cells, as well as for initiation, maintenance, and resolution of tissue repair processes. Macrophages are potent contributors to tissue repair, including through the production of numerous growth factors including, for example, PDGF, IGF-1, TGF-β1, and VEGF-α. In some embodiments, macrophages comprise or express Resolvins (e.g., Resolvin Ds and/or Resolvin Es). In some embodiments, macrophages comprise or express Osteopontin. In some embodiments, macrophages exhibiting an M2 or M0 phenotype are particularly advantageous when used in methods and/or compositions encompassed by the present disclosure.

In some embodiments, a macrophage comprises or is an undifferentiated or M0 macrophage. In certain embodiments, a macrophage comprises or expresses one, two, three, four, five, or six of CD14, CD16, CD64, CD68, CD71, or CCR5. Exposure to various stimuli can induce M0 macrophages to polarize into several distinct populations, which may be identified by macrophage phenotype markers, cytokine production, and/or chemokine secretion.

In some embodiments, a macrophage comprises or is a polarized macrophage. Under classical conditions of activation, M0 macrophages can be exposed to pro-inflammatory signals, such as LPS, IFNγ, and GM-CSF, and polarize into M1 macrophages. Generally, M1 macrophages are associated with pro-inflammatory immune responses, such as Th1 and Th17 T cell responses. Exposure to other stimuli can polarize macrophages into a diverse group of “alternatively activated” or M2 macrophages.

In some embodiments, a macrophage comprises or is an M1 macrophage. In some embodiments, a macrophage expresses one or more markers of M1 macrophages (e.g., one, two, three, four, five, six, seven, eight, nine, 10, 11, 12, 13, 14, 15, 16, 17, or 18 of CD86, CD80, MHC II, IL-1R, TLR2, TLR4, iNOS, SOCS3, CD83, PD-L1, CD69, MHC I, CD64, CD32, CD16, IL1R, a IFIT family member, or an ISG family member).

In some embodiments, a macrophage comprises or is an M2 macrophage (e.g., an M2A, M2B, M2C, or M2D macrophage). An M2A macrophage can be induced by IL-4, IL-13, and/or fungal infection. An M2B macrophage can be induced by IL-1R ligands, an immune complex, and/or LPS. An M2C macrophage can be induced by IL-10 and/or TGFβ. An M2D macrophage can be induced by IL-6 and/or adenosine. In some embodiments, a macrophage expresses one or more markers of M2 macrophages (e.g., one, two, or three of CD206, CD163, or CD209).

In some embodiments, a macrophage comprises at least one upregulated M2 marker and/or at least one downregulated M1 marker. In some embodiments, at least one M2 marker (e.g., CD206, CD163, and/or CD209) is upregulated in a macrophage. In some embodiments, at least one M1 marker (e.g., HLA DR, CD86, CD80, PD-L1, CD83, CD69, MHC I, CD64, CD32, CD16, IL1R, a IFIT family member, and/or an ISG family member) is downregulated in a macrophage.

### Monocytes

The present disclosure, among other things, provides monocytes comprising one or more nucleic acid sequences encoding at least one exogenous fibrolytic agent (e.g., a polypeptide described herein) and/or at least one exogenous anti-inflammatory agent (e.g., a polypeptide described herein). Monocytes are multipotent cells that circulate in the blood, bone marrow, and spleen, and generally do not proliferate when in a steady state. Monocytes can vary in size significantly in the range of about 10-30 μm in diameter. A ratio of nucleus to cytoplasm for a monocyte can range from about 2:1 to about 1:1. Typically, monocytes comprise chemokine receptors and pathogen recognition receptors that mediate migration from blood to tissues, such as during an wound repair or infection. Monocytes can produce inflammatory and/or anti-inflammatory cytokines, take up cells and/or toxic molecules, and differentiate into dendritic cells or macrophages.

In some embodiments, a monocyte comprises or expresses one or more phenotypic markers. Exemplarily phenotypic markers for human monocyte cells include, but are not limited to, CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84. CD85, CD86, CD87, CD89, CD91, CDw92, CD93, CD98, CD101, CD102, CD111, CD112, CD115, CD116, CD119, CDw121b, CDw123, CD127, CDw128, CDw131, CD147, CD155, CD156a, CD157, CD162 CD163, CD164, CD168, CD171, CD172a, CD180, CD206, CD131a1, CD213 2, CDw210, CD226, CD281, CD282, CD284, and CD286. Exemplarily phenotypic markers for mouse monocyte cells include, but are not limited to, CD11a, CD11b, CD16, CD18, CD29, CD31, CD32, CD44, CD45, CD49d, CD115, CD116, Cdw131, CD281, CD282, CD284, CD286, F4/80, and CD49b. In certain embodiments monocytes comprises one, two, or three of CD11b, CD14, or CD16. In certain embodiments, monocytes comprises CD14+ CD16− monocytes, CD14+ CD16+ monocytes, or CD14− CD16+ monocytes.

In some embodiments, a monocyte differentiates into a macrophage. In some embodiments, a monocyte differentiates into a dendritic cell (DC). Monocytes can be differentiated into macrophages or DCs by any technique known in the art. For example, differentiation of monocytes into macrophages can be induced by macrophage colony stimulating factor (M-CSF). Differentiation of monocytes into DCs can be induced by granulocyte-macrophage colony stimulating factor (GM-CSF) in combination with IL-4.

### Dendritic Cells

The present disclosure, among other things, provides dendritic cells (DCs) comprising one or more nucleic acid sequences encoding at least one exogenous fibrolytic agent (e.g., a polypeptide described herein) and/or at least one exogenous anti-inflammatory agent (e.g., a polypeptide described herein). DCs are bone marrow-derived, specialized antigen presenting cells that are involved in initiating immune responses and maintaining tolerance of the immune system to self-antigens. Dendritic cells may be found in both lymphoid and non-lymphoid organs and are generally thought to arise from lymphoid or myeloid lineages.

In some embodiments, a DC comprises or expresses one or more phenotypic markers. Exemplarily phenotypic markers for DCs include, but are not limited to, CD11c, CD83, CD1a, CD1c, CD141, CD207, CLEC9a, CD123, CD85, CD180, CD187, CD205, CD281, CD282, CD284, CD286 and partially CD206, CD207, CD208 and CD209.

Immature DCs can be characterized by a high capacity for antigen capture, but relatively low T cell stimulatory capability Inflammatory mediators promote DC maturation. In some embodiments, a DC comprises or is an immature DC. In other embodiments, a DC comprises or is a mature DC.

### Modified Immune Cell

In some embodiments, a modified immune cell, for example, a modified macrophage, monocyte, or dendritic cell, is generated by expressing at least one exogenous fibrolytic agent (e.g., a polypeptide described herein) and/or at least one exogenous anti-inflammatory agent (e.g., a polypeptide described herein). The present disclosure also provides immune cells comprising a nucleic acid sequence (e.g., an isolated nucleic acid sequence) encoding at least one exogenous fibrolytic agent (e.g., a polypeptide described herein) and/or at least one exogenous anti-inflammatory agent (e.g., a polypeptide described herein), wherein the cell is a monocyte, macrophage or a dendritic cell that expresses at least one exogenous fibrolytic agent, and/or at least one exogenous anti-inflammatory agent.

### Exogenous Fibrolytic Agents

The term “exogenous fibrolytic agent,” as used herein, refers to an exogenous molecule that may be expressed, released, secreted, or delivered, for example, to affect a fibrotic target via modified immune cells described herein (e.g., monocytes, macrophages and/or dendritic cells). A fibrotic target may cause or result from the formation of fibrous tissue and include any biochemical, cellular, or physiological protein associated with fibrosis. A fibrotic target may be associated with any fibrotic condition, disease, or disorder (e.g., a fibrotic condition, disease, or disorder described herein). An exogenous fibrolytic agent can include, but is not limited to, a polypeptide, a fusion protein, a nucleic acid, an antibiotic, an antibody or fragment thereof, an antibody agent or fragment thereof, a growth factor, a cytokine, an enzyme, a synthetic molecule, an organic molecule (e.g., a small molecule), a carbohydrate, a lipid, a hormone, and/or a microsome, or any derivative or variant of any of the foregoing.

In some embodiments, an exogenous fibrolytic agent comprises or is a matrix metallopeptidase (MMP) or a functional fragment thereof. In some embodiments, a MMP comprises or is one or more of MMP-1, MMP-2, MMP-3, MMP-7, MMP-8, MMP-9, MMP-12, MMP-13, MMP-14, MMP-15, MMP-16, MMP-19, MMP-24, and/or TIMP-1, or a functional fragment of any of the foregoing. In some embodiments, an exogenous fibrolytic agent comprises or is a TWEAK polypeptide or a functional fragment thereof.

In some embodiments, an exogenous fibrolytic agent is tethered to an immune cell described herein (e.g., a macrophage, monocyte, or dendritic cell). In some embodiments, an exogenous fibrolytic agent is secreted from an immune cell described herein (e.g., a macrophage, monocyte, or dendritic cell).

In some embodiments, an exogenous fibrolytic agent is tethered to an immune cell described herein (e.g., a macrophage, monocyte, or dendritic cell) by fusion to one or more glycosylphosphatidylinositol (GPI)-anchors, one or more transmembrane receptors or ligands, or one or more hinge domains and/or one or more transmembrane domains. In some embodiments, a hinge domain comprises or is a CD8a, an IgG4, or a CD28 hinge domain. In some embodiments, a transmembrane domain comprises or is a CD8a, CD64, CD32a, CD32c, CD16a, TRL1, TLR2, TLR3, TRL4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, CD3-zeta, FcR γ, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, CD36, or Syk transmembrane domain. In some embodiments, an exogenous fibrolytic agent is tethered to an immune cell described herein (e.g., a macrophage, monocyte, or dendritic cell) by a short linker (e.g., a glycine-rich and/or serine-rich linker). In some embodiments, a linker comprises (GGGGS)n where n=1, 2, 3, 4, or 5, or permuted versions thereof, e.g., (SGGGG)n where n=1, 2, 3, 4, or 5.

In some embodiments, a modified immune cell described herein (e.g., a macrophage, monocyte, or dendritic cell) expressing or comprising at least one exogenous fibrolytic agent decreases or prevents activation of hepatic stellate cells, e.g., relative to an unmodified immune cell of the same type. In some embodiments, a modified immune cell described herein (e.g., a macrophage, monocyte, or dendritic cell) expressing or comprising at least one exogenous fibrolytic agent improves tissue regeneration and/or resolution (e.g., liver tissue regeneration and/or resolution), e.g., relative to an unmodified immune cell of the same type. In some embodiments, a modified immune cell described herein (e.g., a macrophage, monocyte, or dendritic cell) expressing or comprising at least one exogenous fibrolytic agent balances pro-fibrotic and anti-fibrotic immune cell populations (e.g., macrophage populations), e.g., relative to an unmodified immune cell of the same type.

In some embodiments, a modified immune cell described herein (e.g., a macrophage, monocyte, or dendritic cell) expressing or comprising at least one exogenous fibrolytic agent decreases or prevents fibrosis (e.g., liver fibrosis), e.g., relative to an unmodified immune cell of the same type. In some embodiments, a modified immune cell described herein (e.g., a macrophage, monocyte, or dendritic cell) expressing or comprising at least one exogenous fibrolytic agent decreases or prevents tissue injury (e.g., liver tissue injury), e.g., relative to an unmodified immune cell of the same type. In some embodiments, a modified immune cell described herein (e.g., a macrophage, monocyte, or dendritic cell) expressing or comprising at least one exogenous fibrolytic agent decreases a level of alanine transaminase (ALT) and/or aspartate transaminase (AST), e.g., relative to an unmodified immune cell of the same type.

In some embodiments, a modified immune cell described herein (e.g., a macrophage, monocyte, or dendritic cell) expressing or comprising at least one exogenous fibrolytic agent decreases or prevents hepatocyte inflammation (e.g., resulting in a decreased level of NF-kB, IL-1β, IL-2, MCP-1, and/or MIP-1), e.g., relative to an unmodified immune cell of the same type. In some embodiments, a modified immune cell described herein (e.g., a macrophage, monocyte, or dendritic cell) expressing or comprising at least one exogenous fibrolytic agent results in an increased a level or activity of an anti-inflammatory agent (e.g., IL-10, IL-2, and/or CCL18), e.g., relative to an unmodified immune cell of the same type.

In some embodiments, a modified immune cell described herein (e.g., a macrophage, monocyte, or dendritic cell) expressing or comprising at least one exogenous fibrolytic agent decreases or prevents steatosis (e.g., liver steatosis), e.g., relative to an unmodified immune cell of the same type.

### Exogenous Anti-Inflammatory Agents

The term “exogenous anti-inflammatory agent,” as used herein, refers to an exogenous molecule that may be expressed, released, secreted, or delivered, for example, to an inflammatory target via modified immune cells described herein (e.g., monocytes, macrophages and/or dendritic cells). An inflammatory target may cause or result from a biochemical, cellular, or physiological response to noxious stimuli including, but not limited to, injuries, immunologic reactions, infections, altered endogenous substances, defective endogenous pathways, and/or foreign substances. In some embodiments, an inflammatory target is or comprises a protein. An exogenous anti-inflammatory agent can include, but is not limited to, a polypeptide, a fusion protein, a nucleic acid, an antibiotic, an antibody or fragment thereof, an antibody agent or fragment thereof, a growth factor, a cytokine, an enzyme, a synthetic molecule, an organic molecule (e.g., a small molecule), a carbohydrate, a lipid, a hormone, and/or a microsome, or any derivative or variant of any of the foregoing.

In some embodiments, an exogenous anti-inflammatory agent comprises or is a cytokine. In some embodiments, a cytokine comprises or is IL10, IL4, IL13, TGFβ, IL11, IL1RA, IL6, M-CSF, GM-CSF, sTNFRI, sTNFRII, sIL-18BP, and/or IL2-RA. In some embodiments, an exogenous anti-inflammatory agent comprises or is a chemokine. In some embodiments, a chemokine comprises or is CX3CL. In some embodiments, an exogenous anti-inflammatory agent comprises or is a pentraxin. In some embodiments, a pentraxin comprises or is Pentraxin-2.

In some embodiments, an exogenous anti-inflammatory agent is tethered to an immune cell described herein (e.g., a macrophage, monocyte, or dendritic cell). In some embodiments, an exogenous anti-inflammatory agent is secreted from an immune cell described herein (e.g., a macrophage, monocyte, or dendritic cell).

In some embodiments, an exogenous anti-inflammatory agent is tethered to an immune cell described herein (e.g., a macrophage, monocyte, or dendritic cell) by fusion to one or more GPI-anchors, one or more transmembrane receptors or ligands, or one or more hinge domains and/or one or more transmembrane domains. In some embodiments, a hinge domain comprises or is a CD8a, an IgG4, or a CD28 hinge domain. In some embodiments, a transmembrane domain comprises or is a CD8a, CD64, CD32a, CD32c, CD16a, TRL1, TLR2, TLR3, TRL4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, CD3-zeta, FcR γ, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, CD36, or Syk transmembrane domain. In some embodiments, an exogenous anti-inflammatory agent is tethered to an immune cell described herein (e.g., a macrophage, monocyte, or dendritic cell) by a short linker (e.g., a glycine-rich and/or serine-rich linker). In some embodiments, a linker comprises (GGGGS)n where n=1, 2, 3, 4, or 5, or permuted versions thereof, e.g., (SGGGG)n where n=1, 2, 3, 4, or 5.

In some embodiments, a modified immune cell described herein (e.g., a macrophage, monocyte, or dendritic cell) expressing or comprising at least one exogenous anti-inflammatory agent decreases a level of expression of one or more inflammatory agents (e.g., TNFα, IL-6, IL-1β, MCP-1, CCL2, and/or MIP-1), e.g., relative to an unmodified immune cell of the same type. In some embodiments, a modified immune cell described herein (e.g., a macrophage, monocyte, or dendritic cell) expressing or comprising at least one exogenous anti-inflammatory agent decreases inflammatory signaling (e.g., NF-kB signaling), e.g., relative to an unmodified immune cell of the same type. In some embodiments, a modified immune cell described herein (e.g., a macrophage, monocyte, or dendritic cell) expressing or comprising at least one exogenous anti-inflammatory agent increases a level of expression of an anti-inflammatory agent (e.g., IL-10, IL-2, and/or CCL18), e.g., relative to an unmodified immune cell of the same type.

In some embodiments, a modified immune cell described herein (e.g., a macrophage, monocyte, or dendritic cell) expressing or comprising at least one exogenous anti-inflammatory agent decreases or prevents oxidative stress, e.g., relative to an unmodified immune cell of the same type. In some embodiments, a modified immune cell described herein (e.g., a macrophage, monocyte, or dendritic cell) expressing or comprising at least one exogenous anti-inflammatory agent decreases or prevents tissue inflammation (e.g., liver tissue inflammation), e.g., relative to an unmodified immune cell of the same type. In some embodiments, a modified immune cell described herein (e.g., a macrophage, monocyte, or dendritic cell) expressing or comprising at least one exogenous anti-inflammatory agent decreases activation of inflammatory effector cells (e.g. hepatic stellate cells), e.g., relative to an unmodified immune cell of the same type.

### Nucleic Acid Constructs

The present disclosure, among other things, provides nucleic acid molecules comprising one or more nucleic acid sequences encoding at least one exogenous fibrolytic agent (e.g., a polypeptide described herein) and/or at least one exogenous anti-inflammatory agent (e.g., a polypeptide described herein). An immune cell can comprise a nucleic acid molecule (e.g., an exogenous nucleic acid molecule) encoding at least one exogenous fibrolytic agent (e.g., a polypeptide described herein) and/or at least one exogenous anti-inflammatory agent (e.g., a polypeptide described herein).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s). The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “operably linked” or “transcriptional control” refers to functional linkage between a regulatory sequence and a heterologous nucleic acid sequence resulting in expression of the heterologous nucleic acid sequence. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Nucleic acid molecules encoding at least one exogenous fibrolytic agent (e.g., a polypeptide described herein) and/or at least one exogenous anti-inflammatory agent (e.g., a polypeptide described herein) can be a DNA molecule, an RNA molecule, or a combination thereof. In some embodiments, a nucleic acid molecule comprises or is a messenger RNA (mRNA) transcript encoding at least one exogenous fibrolytic agent. In some embodiments, a nucleic acid molecule comprises or is an mRNA transcript encoding at least one exogenous anti-inflammatory agent. In some embodiments, a nucleic acid molecule comprises or is a DNA construct encoding at least one exogenous fibrolytic agent and/or at least one exogenous anti-inflammatory agent.

In some embodiments, all or a fragment of at least one exogenous fibrolytic agent, described herein is encoded by a codon optimized nucleic acid molecule, e.g., for expression in a cell (e.g., a mammalian cell). In some embodiments, all or a fragment of at least one exogenous anti-inflammatory agent, described herein is encoded by a codon optimized nucleic acid molecule, e.g., for expression in a cell (e.g., a mammalian cell). A variety of codon optimization methods are known in the art, e.g., as disclosed in U.S. Pat. Nos. 5,786,464 and 6,114,148, each of which is hereby incorporated by reference in its entirety.

In some embodiments, a vector comprises a nucleic acid molecule encoding at least one exogenous fibrolytic agent (e.g., a polypeptide described herein) and/or at least one exogenous anti-inflammatory agent (e.g., a polypeptide described herein). In some embodiments, a vector comprises a plasmid, viral vector, phagemid, retrotransposon (e.g. piggyback or sleeping beauty), site directed insertion vector (e.g. CRISPR/Cas systems (e.g., CRISPR/Cas systems comprising one or more of Cas9, Cas12a, or C2c2), Zn finger nucleases, and/or TALEN for insertion of a template donor DNA comprising a nucleic acid sequence encoding at least one exogenous fibrolytic agent (e.g., a polypeptide described herein) and/or at least one exogenous anti-inflammatory agent (e.g., a polypeptide described herein), suicide expression vector, or any other vector known in the art. Vectors can be suitable for replication and integration in eukaryotes. Vectors can include expression vectors, replication vectors, probe generation vectors, and sequencing vectors.

A vector can comprise an origin of replication, a promoter sequence (e.g., a constitutive or inducible promoter), and/or convenient restriction endonuclease sites (See, e.g., WO 01/96584; WO 01/29058; and U.S. Pat. No. 6,326,193, each of which are hereby incorporated by reference in their entirety). A vector can also include, e.g., a signal sequence to facilitate secretion, a polyadenylation signal and transcription terminator (e.g., a Bovine Growth Hormone (BGH) polyadenylation signal), an element allowing episomal replication and replication in prokaryotes (e.g., SV40 origin and/or ColE1), elements to allow selection (e.g., an ampicillin resistance gene and/or zeocin marker), and/or reporter genes (e.g., luciferase, beta-galactosidase, chloramphenicol acetyl transferase, secreted alkaline phosphatase, or green fluorescent protein).

Expression of nucleic acids described herein may be achieved by operably linking a nucleic acid encoding at least one exogenous fibrolytic agent (e.g., a polypeptide described herein) to a promoter in an expression vector. Expression of nucleic acids described herein may be achieved by operably linking a nucleic acid encoding at least one exogenous anti-inflammatory agent (e.g., a polypeptide described herein) to a promoter in an expression vector. Exemplary promoters (e.g., constitutive promoters) include, but are not limited to, an elongation factor-1α promoter (EF-1α) promoter, immediate early cytomegalovirus (CMV) promoter, ubiquitin C promoter, phosphoglycerokinase (PGK) promoter, simian virus 40 (SV40) early promoter, mouse mammary tumor virus (MMTV) promoter, human immunodeficiency virus (HIV) long terminal repeat (LTR) promoter, Moloney murine leukemia virus (MoMuLV) promoter, an avian leukemia virus promoter, an Epstein-Barr virus immediate early promoter, a Rous sarcoma virus promoter, an actin promoter, a myosin promoter, a hemoglobin promoter, or a creatine kinase promoter. Examples of inducible promoters include, but are not limited to a metallothionine promoter, a glucocorticoid promoter, a progesterone promoter, and a tetracycline promoter. A vector can also comprise additional promoter elements, e.g., enhancers, to regulate the frequency of transcriptional initiation.

A vector can comprise one or more nucleic acid sequences encoding at least one exogenous fibrolytic agent (e.g., a polypeptide described herein) and/or at least one exogenous anti-inflammatory agent (e.g., a polypeptide described herein) operably linked to one or more tissue specific promoters. In some embodiments, a vector described herein comprises one or more liver specific promoters. In some embodiments, a vector described herein comprises one or more cirrhosis specific promoters. In some embodiments, a vector described herein comprises a CX3CR1 promoter. In some embodiments, a vector described herein comprises an insulin-like growth factor 1 (IGF1). In some embodiments, a vector described herein comprises a CD11B promoter. In some embodiments, a vector (or combination of vectors) may comprise nucleic acid sequences encoding 2, 3, 4, 5, 6, 7, 8, 9, 10 or more fibrolytic agents and/or anti-inflammatory agents.

In some embodiments, a vector comprising one or more nucleic acid sequences encoding at least one exogenous fibrolytic agent (e.g., a polypeptide described herein) and/or at least one exogenous anti-inflammatory agent (e.g., a polypeptide described herein) comprises or is a viral vector. Viral vector technology is well known in the art and is described, e.g., in Sambrook et al., 2012, MOLECULAR CLONING: A LABORATORY MANUAL, volumes 1-4, Cold Spring Harbor Press, NY). Examples of viral vectors include, but are not limited to, adenoviral vectors, adeno-associated viral vectors, or retroviral vectors (e.g., a lentiviral vector or a gammaretroviral vector). In some embodiments, a vector comprises a lentiviral vector (e.g., as described in U.S. Pat. No. 9,149,519 or International Publication No. WO 2017/044487, each of which is hereby incorporated by reference in its entirety).

In some embodiments, a viral vector comprises an adenoviral vector. Adenoviruses are a large family of viruses containing double stranded DNA. They replicate within the nucleus of a host cell, using the host's cell machinery to synthesize viral RNA, DNA and proteins. Adenoviruses are known in the art to affect both replicating and non-replicating cells, to accommodate large transgenes, and to code for proteins without integrating into the host cell genome. In some embodiments, an adenoviral vector comprises an Ad2 vector or an Ad5 vector (e.g., Ad5f35 adenoviral vector, e.g., a helper-dependent Ad5F35 adenoviral vector).

In some embodiments, a viral vector is an adeno-associated virus (AAV) vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. Generally, any AAV serotype may be used to deliver at least one exogenous fibrolytic agent (e.g., a polypeptide described herein) and/or at least one exogenous anti-inflammatory agent (e.g., a polypeptide described herein). In some embodiments, an AAV serotype has a tropism for a particular tissue.

In some embodiments, CRISPR/Cas9 system (or other Cas systems including Cas12a, Cas13, etc) has recently been shown to facilitate high levels of precise genome editing using adeno associated viral (AAV) vectors to serve as donor template DNA during homologous recombination (HR).

In some embodiments, a vector comprises a gammaretroviral vector (e.g., as described in Tobias Maetzig et al., “Gammaretroviral Vectors: Biology, Technology and Application” Viruses. 2011 June; 3(6): 677-713, which is hereby incorporated by reference in its entirety). Exemplary gammaretroviral vectors include Murine Leukemia Virus (MLV), Spleen-Focus Forming Virus (SFFV), and Myeloproliferative Sarcoma Virus (MPSV), and vectors derived therefrom.

In some embodiments, a vector comprises two or more nucleic acid sequences encoding at least one exogenous fibrolytic agent (e.g., a polypeptide described herein) and/or at least one exogenous anti-inflammatory agent (e.g., a polypeptide described herein). In some embodiments, two or more nucleic acid sequences encoding at least two agents (e.g., two or more fibrolytic agents, two or more anti-inflammatory agents, or a combination of fibrolytic and anti-inflammatory agents) are encoded by a single nucleic molecule, e.g., in same frame and as a single polypeptide chain. In some embodiments, two or more agents are separated by one or more cleavage peptide sites (e.g., an auto-cleavage site or a substrate for an intracellular protease). In certain embodiments, a cleavage peptide comprises a porcine teschovirus-1 (P2A) peptide, Thosea asigna virus (T2A) peptide, equine rhinitis A virus (E2A) peptide, foot-and-mouth disease virus (F2A) peptide, or a variant thereof.

### Pharmaceutical Compositions

The present disclosure, among other things, provides pharmaceutical compositions comprising immune cells as described herein (e.g., macrophages, monocytes, or dendritic cells) in combination with one or more pharmaceutically or physiologically acceptable carriers, diluents, or excipients.

When “a therapeutically effective amount, “an immunologically effective amount,” “an anti-immune response effective amount,” or “an immune response-inhibiting effective amount” is indicated, a precise amount of a pharmaceutical composition comprising immune cells as described herein (e.g., macrophages, monocytes, or dendritic cells) can be determined by a physician with consideration of individual differences in age, weight, immune response, and condition of the patient (subject).

Pharmaceutical compositions comprising immune cells as described herein (e.g., macrophages, monocytes, or dendritic cells) may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

Pharmaceutical compositions described herein may be administered in a manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for intravenous infusion or injection. In some embodiments, a pharmaceutical composition described herein is formulated for intramuscular or subcutaneous injection. Pharmaceutical compositions described herein can be formulated for administered by using infusion techniques that are commonly known in immunotherapy (See, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

Pharmaceutical compositions comprising immune cells as described herein may be administered at a dosage of about 10⁴ to about 10⁹ cells/kg body weight (e.g., about 10⁵ to about 10⁶ cells/kg body weight), including all integer values within those ranges). In some embodiments, a dose of immune cells as described herein (e.g., macrophages, monocytes, or dendritic cells) comprises at least about 1×10⁶, about 1.1×10⁶, about 2×10⁶, about 3.6×10⁶, about 5×10⁶, about 1×10⁷, about 1.8×10⁷, about 2×10⁷, about 5×10⁷, about 1×10⁸, about 2×10⁸, about 5×10⁸, about 1×10⁹, about 2×10⁹, or about 5×10⁹ cells. Pharmaceutical compositions described herein may also be administered multiple times at a certain dosage. An optimal dosage and treatment regime for a particular patient can readily be determined by one skilled in the art by monitoring a patient for signs of a disease, disorder, or condition and adjusting treatment accordingly.

It may be desired to administer pharmaceutical compositions comprising immune cells (e.g., macrophages, monocytes, or dendritic cells) as described herein to a subject and then subsequently redraw blood (or have apheresis performed), activate collected immune cells, and reinfuse a subject with activated immune cells. This process can be performed multiple times, e.g., every few weeks. Immune cells (e.g., macrophages, monocytes, or dendritic cells) can be activated from blood draws of from about 10 cc to about 400 cc. In some embodiments, immune cells (e.g., macrophages, monocytes, or dendritic cells) are activated from blood draws of about 20 cc, about 30 cc, about 40 cc, about 50 cc, about 60 cc, about 70 cc, about 80 cc, about 90 cc, or about 100 cc. Without being bound by theory, methods comprising multiple blood draw and reinfusions as described herein may select for certain immune cell populations.

In some embodiments, pharmaceutical compositions comprising immune cells as described herein (e.g., macrophages, monocytes, or dendritic cells) are administered in combination with (e.g., before, simultaneously, or following) a second therapy. For example, a second therapy can include, but is not limited to farnesoid X receptor agonist, a stearoyl CoA desaturase inhibitor, a CCR2/CCR5 chemokine antagonist, a PPAR agonist, a caspase inhibitor, a galectin-3 inhibitor, an acetyl CoA carboxylase inhibitor, a lysyl oxidase-2 (LOX-2) antagonist, TGFβ antagonist, and/or TIMP antagonist. A dosage of any aforementioned therapy to be administered to a subject will vary with a disease, disorder, or condition being treated and based on a specific subject. Scaling of dosages for human administration can be performed according to art-accepted practices.

### Methods of Treatment

The present disclosure, among other things, provides methods of treating a disease, disorder, or condition (e.g., a disease, disorder, or condition described herein) in a subject comprising delivering a pharmaceutical composition comprising immune cells as described herein (e.g., macrophages, monocytes, or dendritic cells). In some embodiments, a therapeutically effective amount of a pharmaceutical composition described herein is administered to a subject having a disease, disorder, or condition.

A subject to be treated with methods described herein can be a mammal, e.g., a primate, e.g., a human (e.g., a subject having, or at risk of having, a disease, disorder, or condition described herein). In some embodiments, immune cells (e.g., macrophages, monocytes, or dendritic cells) may be autologous, allogeneic, or xenogeneic with respect to a subject. Pharmaceutical compositions as described herein can be administered to a subject in accordance with a dosage regimen described herein, alone or in combination with one or more therapeutic agents, procedures, or modalities.

Administration of pharmaceutical compositions described herein may be carried out in any convenient manner (e.g., injection, ingestion, transfusion, inhalation, implantation, or transplantation). In some embodiments, a pharmaceutical compositions described herein is administered by injection or infusion. Pharmaceutical compositions described herein may be administered to a subject transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

Fibrosis

Modified immune cells described herein can be administered to improve one or more symptoms of or reduce fibrosis in a subject, e.g., to treat or prevent a fibrotic disease, disorder, or condition in a subject. Methods can include administering modified immune cells described herein to a subject in need thereof, in an amount sufficient to decrease one or more symptoms of or prevent fibrosis in the subject. Modified immune cells described herein can be administered to improve tissue repair, e.g., in a subject with a fibrotic disease, disorder, or condition. In some embodiments, a subject has fibrosis or has been diagnosed with a fibrotic disease, disorder, or condition. In some embodiments, a subject has not received prior treatment with modified immune cells described herein (e.g., a naïve subject).

In some embodiments, modified immune cells described herein are for use as a medicament in treating (e.g., reversing, reducing, ameliorating, or preventing) fibrosis in a subject (e.g., a subject with a fibrotic disease, disorder, or condition). In some embodiments, modified immune cells described herein are for use in the manufacture of a medicament for treating (e.g., reversing, reducing or ameliorating one or more symptoms of, or preventing) fibrosis in a subject (e.g., a subject with a fibrotic disease, disorder, or condition).

In some embodiments, administration of modified immune cells described herein to a subject delays onset of or reduces one or more of: the formation or deposition of tissue fibrosis; the size, cellularity, composition, or cellular content of a fibrotic lesion; the collagen and/or hydroxyproline content of a fibrotic lesion; expression or activity of a fibrogenic protein; fibrosis associated with an inflammatory response; and/or weight loss associated with fibrosis as compared to a subject that did not receive such administration. In some embodiments, reducing fibrosis increases survival of a subject as compared to a subject that did not receive such administration.

Exemplary fibrotic diseases, disorders, or conditions include systemic diseases, disorders, or conditions (e.g., systemic sclerosis, multifocal fibrosclerosis, sclerodermatous chronic graft-versus-host disease, nephrogenic systemic fibrosis, or scleroderma) and organ-specific diseases, disorders, or conditions (e.g., liver, lung, heart, kidney, pancreas, skin, and/or nervous system fibrosis). In some embodiments, a fibrotic disease, disorder, or condition comprises or is a hyperproliferative fibrotic disease, e.g., a non-cancerous fibrotic disease.

In some embodiments, a fibrotic disease, disorder, or condition comprises or is a liver, lung, heart, vasculature, kidney, pancreas, skin, gastrointestinal, bone marrow, hematopoietic tissue, nervous system, and/or eye fibrotic disease, disorder, or condition. In some embodiments, a fibrotic disease, disorder, or condition affects tissue comprising tendon, cartilage, skin (e.g., skin epidermis and/or endodermis), cardiac tissue, vascular tissue (e.g., artery and/or vein), pancreatic tissue, lung tissue, kidney tissue, uterine tissue, ovarian tissue, neural tissue, testicular tissue, peritoneal tissue, colon, small intestine, biliary tract, gut, bone marrow, hematopoietic tissue, and/or eye tissue (e.g., retinal tissue).

In some embodiments, a fibrotic disease, disorder, or condition comprises or is a liver fibrotic disease, disorder, or condition. Modified immune cells described herein can be administered to improve liver function, e.g., in a subject with a liver fibrotic disease, disorder, or condition. In some embodiments, a liver fibrotic disease, disorder, or condition comprises or is a fatty liver disease, disorder, or condition. In some embodiments, a fatty liver disease, disorder, or condition comprises or is non-alcoholic fatty liver disease (NAFL) (e.g., non-alcoholic fatty liver disease (NAFLD) or non-alcoholic steatohepatitis (NASH)) or alcoholic liver disease (e.g., alcoholic fatty liver disease (AFLD) or alcoholic steatohepatitis (ASH)). In some embodiments, a subject has cirrhosis, hepatocarcinoma, an increased risk of liver failure, an increased risk of death, metabolic syndrome, type 2 diabetes, Hepatitis B infection (HBV), mechanical trauma, biliary obstruction, autoimmune hepatitis, iron overload, Hepatitis B infection (HBV), and/or Hepatitis C infection (HCV).

In some embodiments, a fibrotic disease, disorder, or condition comprises or is a lung fibrotic disease, disorder, or condition. Modified immune cells described herein can be administered to improve lung function, e.g., in a subject with a lung fibrotic disease, disorder, or condition. In some embodiments, a lung fibrotic disease, disorder, or condition comprises or is pulmonary fibrosis, idiopathic pulmonary fibrosis (IPF), usual interstitial pneumonitis (UIP), interstitial lung disease, cryptogenic fibrosing alveolitis (CFA), bronchiectasis, and/or scleroderma lung disease. In some embodiments, lung fibrosis is associated with an occupational hazard, an environmental pollutant, cigarette smoking, an autoimmune connective tissue disorders (e.g., rheumatoid arthritis, scleroderma, or systemic lupus erythematosus (SLE)), a connective tissue disorder (e.g., sarcoidosis), and/or or an infectious disease.

In some embodiments, a fibrotic disease, disorder, or condition comprises or is a kidney fibrotic disease, disorder, or condition. Modified immune cells described herein can be administered to improve kidney function, e.g., in a subject with a kidney fibrotic disease, disorder, or condition. In some embodiments, a kidney fibrotic disease, disorder, or condition comprises or is renal fibrosis (e.g., chronic kidney fibrosis), nephropathies associated with fibrosis (e.g., diabetic nephropathy), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, and/or HIV-associated nephropathy. In some embodiments, kidney fibrosis is associated with exposure to a toxin, an irritant, or a chemotherapeutic agent.

In some embodiments, a fibrotic disease, disorder, or condition comprises or is a heart fibrotic disease, disorder, or condition. Modified immune cells described herein can be administered to improve heart function, e.g., in a subject with a heart fibrotic disease, disorder, or condition. In some embodiments, a heart fibrotic disease, disorder, or condition comprises or is myocardial fibrosis. In some embodiments, myocardial fibrosis is associated with radiation myocarditis, surgical procedure complications (e.g., myocardial post-operative fibrosis), infectious diseases (e.g., Chagas disease), granulomatous, metabolic storage disorders (e.g., cardiomyopathy or hemochromatosis), developmental disorders (e.g., endocardial fibroelastosis), arteriosclerotic, and/or exposure to toxins or irritants (e.g., drug-induced cardiomyopathy, drug-induced cardiotoxicity, or alcoholic cardiomyopathy). In some embodiments, myocardial fibrosis is associated with myocardial sarcoidosis, myocardial infarction, and/or congestive heart failure.

In some embodiments, a fibrotic disease, disorder, or condition comprises or is an eye fibrotic disease, disorder, or condition. Modified immune cells described herein can be administered to improve eye function, e.g., in a subject with an eye fibrotic disease, disorder, or condition. In some embodiments, an eye fibrotic disease, disorder, or condition comprises or is glaucoma, macular degeneration (e.g., age-related macular degeneration), macular edema (e.g., diabetic macular edema), retinopathy (e.g., diabetic retinopathy), and/or dry eye disease.

In some embodiments, a fibrotic disease, disorder, or condition comprises or is a skin fibrotic disease, disorder, or condition. Modified immune cells described herein can be administered to improve skin function, e.g., in a subject with a skin fibrotic disease, disorder, or condition. In some embodiments, a skin fibrotic disease, disorder, or condition comprises or is skin fibrosis (e.g., hypertrophic scarring and/or keloid scars), scleroderma of the skin, and/or nephrogenic systemic fibrosis.

In some embodiments, a fibrotic disease, disorder, or condition comprises or is a gastrointestinal tract fibrotic disease, disorder, or condition. Modified immune cells described herein can be administered to improve gastrointestinal tract function, e.g., in a subject with a gastrointestinal tract fibrotic disease, disorder, or condition. In some embodiments, a gastrointestinal tract fibrotic disease, disorder, or condition comprises or is radiation-induced gut fibrosis, fibrosis associated with a foregut inflammatory disorder (e.g., Barrett's esophagus or chronic gastritis), and/or fibrosis associated with a hindgut inflammatory disorder (e.g., inflammatory bowel disease, ulcerative colitis, or Crohn's disease).

In some embodiments, a fibrotic disease, disorder, or condition comprises or is a chronic fibrotic disease, disorder, or condition. In some embodiments, a fibrotic disease, disorder, or condition is associated with an inflammatory disease, disorder, or condition. In some embodiments, a fibrotic disease, disorder, or condition comprises or is osteomyelitis (e.g., chronic osteomyelitis). In some embodiments, a fibrotic disease, disorder, or condition comprises or is an amyloidosis (e.g., amyloidosis associated with chronic osteomyelitis).

### Inflammation

Modified immune cells described herein can be administered to improve or reduce inflammation in a subject, e.g., to treat or prevent an inflammatory disease, disorder, or condition in a subject (e.g., one or more symptoms of an inflammatory disease, disorder, or condition). Methods can include administering modified immune cells described herein to a subject in need thereof, in an amount sufficient to decrease or prevent inflammation in the subject. In some embodiments, a subject has inflammation or has been diagnosed with an inflammatory disease, disorder, or condition. In some embodiments, a subject has not received prior treatment with modified immune cells described herein (e.g., a naïve subject).

In some embodiments, modified immune cells described herein are for use as a medicament in treating (e.g., reversing, reducing, ameliorating, or preventing) inflammation in a subject (e.g., a subject with an inflammatory disease, disorder, or condition). In some embodiments, modified immune cells described herein are for use in the manufacture of a medicament for treating (e.g., reversing, reducing or ameliorating one or more symptoms of, or preventing) inflammation in a subject (e.g., a subject with an inflammatory disease, disorder, or condition).

In some embodiments, an inflammatory disease, disorder, or condition comprises or is a systemic disease, disorder, or condition. In some embodiments, an inflammatory disease, disorder, or condition comprises or is a local disease, disorder, or condition. In some embodiments, an inflammatory disease, disorder, or condition comprises or is an acute disease, disorder, or condition. In some embodiments, an inflammatory disease, disorder, or condition comprises or is a chronic disease, disorder, or condition.

Exemplary inflammatory diseases, disorders, or conditions include, but are not limited to, systemic diseases, disorders, or conditions (e.g., chronic systemic inflammation, Behçet disease, sarcoidosis, systemic lupus erythematosus, juvenile idiopathic arthritis, scleroderma, Sjögren syndrome, and/or sepsis) and organ-specific diseases, disorders, or conditions (e.g., liver, lung, heart, gut, kidney, and/or pancreas inflammation). In some embodiments, an inflammatory disease, disorder, or condition comprises or is a liver, gastrointestinal tract, lung, skin, cardiovascular system, nervous system, kidney, pancreas, joint, eye, and/or an endocrine system inflammatory disease, disorder, or condition. In some embodiments, an inflammatory disease, disorder, or condition comprises or is an autoimmune disorder.

In some embodiments, an inflammatory disease, disorder, or condition comprises or is a liver inflammatory disease, disorder, or condition. In some embodiments, a liver inflammatory disease, disorder, or condition comprises or is a fatty liver disease, disorder, or condition (e.g., NAFLD (e.g., NASH) or AFLD (e.g., ASH)). In certain embodiments, a liver inflammatory disease, disorder, or condition comprises or is cirrhosis (e.g., primary biliary cirrhosis or primary sclerosing cholangitis), hepatitis (e.g., hepatitis from a viral infection, an autoimmune response, a drug treatment, a toxins, and/or an environmental agent), and/or biliary atresia.

In some embodiments, an inflammatory disease, disorder, or condition comprises or is a gastrointestinal tract inflammatory disease, disorder, or condition. In certain embodiments, a gastrointestinal tract inflammatory disease, disorder, or condition comprises or is colitis (e.g., ulcerative colitis, pseudomembranous colitis, microscopic colitis, indeterminatal colitis, ischemic colitis, radiation colitis, and/or collagenous colitis), Crohn's disease, leaky gut syndrome, irritable bowel syndrome (IBS), Barrett's esophagus, intestinal inflammation, chronic gastritis, distal proctitis, enteritis, enterocolitis, gastritis, gastroenteritis, cholangitis, ileitis, constipation, diarrhea; indigestion or non-ulcer dyspepsia, diverticulosis, and/or polyps.

In some embodiments, an inflammatory disease, disorder, or condition comprises or is a lung inflammatory disease, disorder, or condition. In some embodiments, the lung inflammatory disease, disorder, or condition comprises or is asthma, COPD, adult respiratory distress syndrome (ARDS), bronchitis (e.g., chronic bronchitis), bronchiolitis, pulmonary inflammation, pulmonary fibrosis, cystic fibrosis, pneumonitis (e.g., hypersensitivity pneumonitis, usual interstitial pneumonitis (UIP), pneumonia, extrinsic allergic alveolitis, asbestosis, silicosis, bronchiectasis, berylliosis, talcosis, pneumoconiosis, lung sarcoidosis, and/or pleuritis.

In some embodiments, an inflammatory disease, disorder, or condition comprises or is a skin inflammatory disease, disorder, or condition. In some embodiments, a skin inflammatory disease, disorder, or condition comprises or is psoriasis, eczema, dermatitis (e. g., eczematous dermatitides, topic or seborrheic dermatitis, allergic or irritant contact dermatitis, eczema craquelee, photoallergic dermatitis, phototoxicdermatitis, phytophotodermatitis, radiation dermatitis, or stasis dermatitis), an ulcer, ichthyoses, epidermolysis bullosae, a hypertrophic scar, a keloid, inflammatory dermatosis, photoaging, cutaneous atrophy, inflammatory dermatosis, dermatomyositis; pemphigus.

In some embodiments, an inflammatory disease, disorder, or condition comprises or is a cardiovascular system inflammatory disease, disorder, or condition. In some embodiments, a cardiovascular system inflammatory disease, disorder, or condition comprises or is atherosclerosis, coronary infarct damage, peripheral vascular disease, myocarditis, vasculitis, revascularization of stenosis, myocarditis, pericarditis, vascular disease associated with Type II diabetes, endocarditis, a cholesterol-related metabolic disorder, oxygen free radical injury, and/or ischemia.

In some embodiments, an inflammatory disease, disorder, or condition comprises or is a nervous system inflammatory disease, disorder, or condition. In some embodiments, a nervous system inflammatory disease, disorder, or condition comprises or is a neurodegenerative disease (e.g., Alzheimer's disease or dementia), multiple sclerosis, encephalitis (e.g., encephalitis with inflammatory edema), depression, attention deficit disorder (ADD), Parkinson's disease, schizophrenia, and/or a neuropathy (e.g., peripheral neuropathy).

In some embodiments, an inflammatory disease, disorder, or condition comprises or is a kidney inflammatory disease, disorder, or condition. In some embodiments, a kidney inflammatory disease, disorder, or condition comprises or is a nephritis, nephritis secondary to Wegener's disease, acute renal failure secondary to acute nephritis, post-obstructive syndrome, tubular ischemia, pyelonephritis glomerulosclerosis, membranous neuropathy, and/or renal arteriosclerosis. In some embodiments, nephritis comprises or is glomerulonephritis, interstitial nephritis, and/or lupus nephritis.

In some embodiments, an inflammatory disease, disorder, or condition comprises or is a pancreas inflammatory disease, disorder, or condition. In some embodiments, a pancreas inflammatory disease, disorder, or condition comprises or is pancreatitis (e.g., acute or chronic pancreatitis).

In some embodiments, an inflammatory disease, disorder, or condition comprises or is a joint inflammatory disease, disorder, or condition. In some embodiments, a joint inflammatory disease, disorder, or condition comprises or is rheumatoid arthritis, rheumatoid spondylitis, juvenile rheumatoid arthritis, osteoarthritis, gouty arthritis, psoriatic arthritis, lupus-associated arthritis, ankylosing spondylitis, spondyloarthrosis, degenerative arthritis, and/or synovitis.

In some embodiments, an inflammatory disease, disorder, or condition comprises or is an eye inflammatory disease, disorder, or condition. In some embodiments, an eye inflammatory disease, disorder, or condition comprises or is uveitis, iritis, optic neuritis, conjunctivitis, scleritis, iritis, keratoconjunctivitis sicca, blepharitis, age-related macular degeneration (AMD), dry eye syndrome, optic nerve damage, diabetic retinopathy, corneal inflammation, and/or retinal inflammation.

In some embodiments, an inflammatory disease, disorder, or condition comprises or is an endocrine system disease, disorder, or condition. In some embodiments, an endocrine system inflammatory disease, disorder, or condition comprises or is autoimmune thyroiditis (Hashimoto's disease), adrenal cortex inflammation (e.g., acute adrenal cortex inflammation or chronic adrenal cortex inflammation), and/or Type I diabetes mellitus.

In some embodiments, an inflammatory disease, disorder, or condition comprises or is an autoimmune disease, disorder, or condition. In some embodiments, an autoimmune disease, disorder, or condition comprises or is arthritis (e.g., rheumatoid arthritis, juvenile rheumatoid arthritis, osteoarthritis, psoriatic arthritis, lupus-associated arthritis, and/or ankylosing spondylitis), autoimmune thyroiditis, scleroderma, lupus, systemic lupus erythematosus (SLE), HIV, Sjogren's syndrome, vasculitis, multiple sclerosis, dermatitis (e.g., atopic dermatitis or eczematous dermatitis), myasthenia gravis, IBD, Crohn's disease, colitis, an acute inflammatory condition (e.g., endotoxemia, septicaemia, or toxic shock syndrome), a transplant rejection, allergy (e.g., asthma, allergic rhinitis, eczema, allergic contact dermatitis, or allergic conjunctivitis), Wegener's gramilornatosis, angiitis, polymyalgia rheumatica (PMR), tendonitis, bursitis, an immediate hypersensitivity reaction (e.g., asthma, hay fever, cutaneous allergies, or acute anaphylaxis), acute disseminated encephalomyelitis, Sorgen's disease, Addison's disease, heart disease, osteoporosis, alopecia universalis, antiphospholipid antibody syndrome, autoimmune hemolytic anemia, pernicious anemia, autoimmune hepatitis, Bullous pemphigoid, endometriosis, Goodpasture's syndrome, hidradenitis suppurativa, idiopathic thrombocytopenic purpura, interstitial cystitis, morphea, neuromyotonia, temporal arteritis, vasculitis, and/or vitiligo.

In some embodiments, an inflammatory disease, disorder, or condition comprises or is a wound. In certain embodiment, a wound comprises or is an acute wound, a chronic wound, an open wound, a closed wound, an infected wound, an external wound, an internal wound, and/or a hemorrhage.

In some embodiments, an inflammatory disease, disorder, or condition comprises or is ischemia. In some embodiments, ischemia comprises or is cardiac ischemia, bowel ischemia, brain ischemia (e.g., stroke), limb ischemia, mesenteric ischemia, cutaneous ischemia. In some embodiments, an inflammatory disease, disorder, or condition comprises or is ischemia reperfusion injury, post-perfusion syndrome, and/or transient ischemia of an organ (e.g., gastrointestinal tract, bladder, or heart).

In some embodiments, an inflammatory disease, disorder, or condition comprises or is sepsis. In some embodiments, species comprise or is sepsis resulting from pneumonia, abdominal infection, kidney infection, and/or bloodstream infection. In some embodiments, sepsis comprises or is sepsis syndrome, gram positive sepsis, gram negative sepsis, culture negative sepsis, fungal sepsis, and/or urosepsis.

In some embodiments, an inflammatory disease, disorder, or condition comprises an infectious disease. In some embodiments, an infectious disease comprises or is malaria, pneumonia, African trypanosomiasis, tuberculosis, HIV, human cytomegalovirus (HCMV), herpes virus infection, influenza (e.g., influenzavirus A, influenzavirus B, or influenzavirus C), Epstein-Barr Virus infection, Chagas disease, bacterial, trichinosis, or fungal myocarditis, meningitis, Legionella, hepatitis (e.g., chronic active hepatitis), pneumonia, Clostridium difficile infection, small intestine bacterial overgrowth (SIBO), Dengue hemorrhagic fever, lyme disease, meningococcemia, necrotizing fascilitis, necrotizing enterocolitis, leprosy, streptococcal myositis, infectious colitis, mycoses (e.g., Candida albicans infection), Vancomycin-resistant enterococci (VRE) infection, pneumonia epiglottitis, peritonitis, hemolytic uremic syndromic, toxic shock syndrome, Pneumocystis carinii, Campylobacter jejuni infection, Helicobacter pylori infection, viral encephalitis, septic arthritis, gas gangrene, pelvic inflammatory disease, Mycobacterium avium-intracellulare infection, orchitis, and/or virus-associated hemophagocytic syndrome (VAHS).

### Methods of Immune Cell Modification

The present disclosure, among other things, provides methods for modifying an immune cell (e.g., a monocyte, macrophage, or dendritic cell) comprising delivering a nucleic acid construct comprising one or more nucleic acid sequences encoding at least one exogenous fibrolytic agent (e.g., a polypeptide described herein) and/or at least one exogenous anti-inflammatory agent (e.g., a polypeptide described herein).

A nucleic acid construct comprising one or more nucleic acid sequences encoding at least one exogenous fibrolytic agent (e.g., a polypeptide described herein) and/or at least one exogenous anti-inflammatory agent (e.g., a polypeptide described herein) can be introduced into an immune cell (e.g., a monocyte, macrophage, or dendritic cell) by physical, chemical, or biological methods. Physical methods for introducing a nucleic acid construct as described herein into an immune cell (e.g., a monocyte, macrophage, or dendritic cell) can comprise electroporation, calcium phosphate precipitation, lipofection, particle bombardment, microinjection, or a combination thereof. A nucleic acid construct can be introduced into immune cells using commercially available methods, including electroporation (Amaxa Nucleofector-II® (Amaxa Biosystems, Cologne, Germany), ECM 830 BTX (Harvard Instruments, Boston, Mass.) Gene Pulser II® (BioRad, Denver, Colo.), or Multiporator® (Eppendort, Hamburg Germany)). A nucleic acid construct can also be introduced into immune cells using mRNA transfection, e.g., cationic liposome-mediated transfection, lipofection, polymer encapsulation, peptide-mediated transfection, or biolistic particle delivery systems, such as “gene guns” (See, e.g., Nishikawa, et al. Hum Gene Ther., 12(8):861-70 (2001), which is hereby incorporated by reference in its entirety).

Biological methods for introducing a nucleic acid construct as described herein into an immune cell (e.g., a monocyte, macrophage, or dendritic cell) include use of DNA and RNA vectors. Viral vectors, and especially retroviral vectors, have become widely used for inserting genes into mammalian cells (e.g., human cells). Viral vectors can also be derived from lentivirus, poxviruses, herpes simplex virus I, adenoviruses (e.g. Adf535), or adeno-associated viruses (See, e.g., U.S. Pat. Nos. 5,350,674 and 5,585,362, which are hereby incorporated by reference in their entirety). Retroviral vectors, such as lentivirus, are suitable tools to achieve long-term gene transfer that allow for long-term, stable integration of a transgene and its propagation in daughter cells.

Immune cells described herein (e.g., macrophages, monocytes, or dendritic cells) can be refractory to lentiviral transduction because of expression of a restriction factor, SAMHD1, which depletes nucleotide triphosphates available for reverse transcription. For example, SAMHD1 can restrict replication of human immunodeficiency virus type 1 (HIV-1) by depleting an intracellular pool of deoxynucleoside triphosphates. Viral protein X (Vpx), an accessory protein associated with simian immunodeficiency virus (SIV) and HIV-2, induces degradation of SAMHD1. In some embodiments, delivery of (a) a viral vector comprising one or more nucleic acid sequences encoding at (i) at least one exogenous fibrolytic agent described herein, and/or (ii) at least one exogenous anti-inflammatory agent described herein and (b) at least one Vpx protein can increase transfection of immune cells describes herein (e.g., macrophages, monocytes, or dendritic cells), e.g., relative to the same type of immune cell comprising (i) at least one exogenous fibrolytic agent, and/or (ii) at least one exogenous anti-inflammatory agent and not delivered at least one Vpx protein.

In some embodiments, Vpx lentivirus can lead to genomic integration of at least one exogenous fibrolytic agent, and/or (ii) at least one exogenous anti-inflammatory agent, thereby enabling long-term, permanent expression of (i) at least one exogenous fibrolytic agent, and/or (ii) at least one exogenous anti-inflammatory agent. Vpx-lentivirus transduced macrophages are not phenotypically impacted by viral transduction—a finding that enables the production of modified macrophages as described herein with phenotypic plasticity. While other viral vectors (such as Ad5f35) induce an M1, pro-inflammatory phenotype, Vpx-lentivirus does not have an impact on the M1/M2 phenotype—enabling an M0 modified macrophage product that may not have pro-inflammatory functions/toxicities at baseline. Vpx-lentivirus transduced macrophages retain phenotypic plasticity and may be further polarized to an M1 or M2 phenotype with cytokines, agonists, peptides, culture media, and other factors. Vpx-lentivirus transduced macrophages can be exposed to pro-inflammatory signals (e.g., one or more pro-inflammatory cytokines, e.g., one or more of LPS, IFNa, IFNb, IFNγ, CpG, CD40L, GM-CSF, TNFa, IL-6, or a STING ligand (STING-L)) and polarize into M1 macrophages. Vpx-lentivirus transduced macrophages can be exposed to immunosuppressive signals (e.g., one or more immunosuppressive cytokines (e.g., one or more of IL-4, IL-10, IL-13, or TGFb) and/or one or both of at least one prostaglandin or at least corticosteroid) and polarize into M2 macrophages.

In some embodiments, a lentiviral vector is packaged with a Vpx protein (e.g., as described in International Publication No. WO 2017/044487, which is hereby incorporated by reference in its entirety). In some embodiments, Vpx comprises a virion-associated protein (e.g., an accessory protein for viral replication). In some embodiments, a Vpx protein is encoded by human immunodeficiency virus type 2 (HIV-2). In some embodiments, a Vpx protein is encoded by simian immunodeficiency virus (SIV). In some embodiments, an immune cell as described herein (e.g., a monocyte, macrophage, or dendritic cell) is transfected with a lentiviral vector packaged with a Vpx protein. In some embodiments, Vpx inhibits at least one antiviral factor of an immune cell as described herein (e.g., a monocyte, macrophage, or dendritic cell). In some embodiments, a lentiviral vector packaged with a Vpx protein exhibits increased transfection efficiency of an immune cell as described herein (e.g., a monocyte, macrophage, or dendritic cell), e.g., relative to a lentiviral vector not packaged with a Vpx protein. In some embodiments, an immune cell as described herein (e.g., a monocyte, macrophage, or dendritic cell) is one or both of electroporated or transfected with at least one VPX mRNA prior to transfection with a viral vector (e.g., an adenoviral vector, e.g., an Ad2 vector or an Ad5 vector (e.g., Ad5f35 adenoviral vector, e.g., a helper-dependent Ad5F35 adenoviral vector)).

Chemical means for introducing a nucleic acid construct as described herein into an immune cell (e.g., a monocyte, macrophage, or dendritic cell) include colloidal dispersion systems, macromolecule complexes, nanocapsules, microspheres, beads, and lipid-based systems (e.g., oil-in-water emulsions, micelles, mixed micelles, nanoparticles, liposomes, and lipofectamine-nucleic acid complexes).

An exemplary system for delivery of a nucleic acid construct as described herein is a lipid-based system. A nucleic acid construct as described herein may be encapsulated in an aqueous interior of a liposome, interspersed within a lipid bilayer, attached to a liposome via a linking molecule, entrapped in a liposome, complexed with a liposome, dispersed in a solution or suspension comprising a lipid, mixed with a lipid, complexed with a micelle, or otherwise associated with a lipid. Lipids for use in methods described herein may be naturally occurring or synthetic lipids. Lipids can also be obtained from commercial sources. For example, dimyristyl phosphatidylcholine can be obtained from Sigma (St. Louis, MO); dicetyl phosphate can be obtained from K & K Laboratories (Plainview, NY); cholesterol can be obtained from Calbiochem-Behring; and dimyristyl phosphatidylglycerol can be obtained from Avanti Polar Lipids, Inc. (Birmingham, AL.). Stock solutions of lipids in chloroform or chloroform/methanol can be stored at about −20° C.

A variety of assays may be performed to confirm presence of a nucleic acid construct as described herein in an immune cell (e.g., a monocyte, macrophage, or dendritic cell). For example, such assays include molecular biological assays well known to those of skill in the art, such as Southern and Northern blotting, RT-PCR, and PCR; and biochemical assays, such as detecting the presence or absence of a particular peptide, e.g., by immunological means (ELISAs and Western blots).

### Treatment and Culturing of Immune Cells During Modification

In some embodiments, an immune cell (e.g., a monocyte, macrophage, or dendritic cell) is treated and/or cultured during immune cell modification. In some embodiments, methods of the present disclosure comprise treating an immune cell (e.g., a monocyte, macrophage, or dendritic cell) with a modulator of a pathway activated by in vitro transcribed mRNA. In vitro transcribed (IVT) mRNA is recognized by various endosomal innate immune receptors (Toll-like receptor 3 (TLR3), TLR7 and TLR8) and cytoplasmic innate immune receptors (protein kinase RNA-activated (PKR), retinoic acid-inducible gene I protein (RIG-I), melanoma differentiation-associated protein 5 (MDA5) and 2′-5′-oligoadenylate synthase (OAS)). Signaling through these different pathways results in inflammation associated with type 1 interferon (IFN), tumor necrosis factor (TNF), interleukin-6 (IL-6), IL-12 and the activation of cascades of transcriptional programs. Overall, these create a pro-inflammatory microenvironment poised for inducing specific immune responses. Moreover, downstream effects such as slow-down of translation by eukaryotic translation initiation factor 2α (eIF2α) phosphorylation, enhanced RNA degradation by ribonuclease L (RNaseL), and overexpression and inhibition of replication of self-amplifying mRNA are of relevance for the pharmacokinetics and pharmacodynamics of IVT mRNA.

In some embodiments, a modulator of a pathway activated by in vitro transcribed mRNA comprises an RNase inhibitor. In some embodiments, a modulator of a pathway activated by in vitro transcribed mRNA comprises an RNaseL, RNase T2 or RNase1 inhibitor. In some embodiments, a modulator of a pathway activated by in vitro transcribed mRNA comprises an RNaseL inhibitor. In some embodiments, an RNaseL inhibitor comprises sunitinib. In some embodiments, an RNaseL inhibitor comprises ABCE1.

In some embodiments, treating an immune cell (e.g., a monocyte, macrophage, or dendritic cell) with an RNaseL inhibitor increases mRNA stability in a modified immune cell relative to mRNA stability in a modified immune cell of the same type that was not treated with an RNaseL inhibitor. In some embodiments, treating an immune cell (e.g., a monocyte, macrophage, or dendritic cell) with an RNaseL inhibitor increases exogenous fibrolytic agent expression in a modified immune cell relative to exogenous fibrolytic agent expression in a modified immune cell of the same type that was not treated with an RNaseL inhibitor. In some embodiments, treating an immune cell (e.g., a monocyte, macrophage, or dendritic cell) with an RNaseL inhibitor increases exogenous anti-inflammatory agent expression in a modified immune cell relative to exogenous anti-inflammatory agent expression in a modified immune cell of the same type that was not treated with an RNaseL inhibitor. In some embodiments, treating an immune cell (e.g., a monocyte, macrophage, or dendritic cell) with an RNaseL inhibitor increases effector activity in a modified immune cell relative to effector activity in a modified immune cell of the same type that was not treated with an RNaseL inhibitor.

In some embodiments of the present disclosure, a step of treating an immune cell (e.g., a monocyte, macrophage, or dendritic cell) occurs before a step of delivering an mRNA to the immune cell.

In some embodiments, methods of the present disclosure comprise a step of culturing an immune cell (e.g., a monocyte, macrophage, or dendritic cell) with a cytokine or immune stimulating recombinant protein. In some embodiments, a cytokine comprises IFN-α, IFN-β, IFN-γ, TNFα, IL-6, STNGL, LPS, a CD40 agonist, a 4-1BB ligand, recombinant 4-1BB, a CD19 agonist, a TLR agonist (e.g., TLR-1, TLR-2, TLR-3, TLR-4, TLR-5, TLR-6, TLR-7, TLR-8 or TLR-9), TGF-β (e.g., TGF-β1, TGF-β2, or TGF-β3), IL-1β, IL-2, IL-3, IL-4, IL-5, IL-6, IL-7, IL-9, IL-10, IL-12, IL-13, IL-14, IL-15, IL-16, IL-17, IL-18, IL-20, granulocyte-macrophage colony-stimulating factor (GM-CSF), granulocyte colony-stimulating factor (G-CSF), Leukemia inhibitory factor (LIF), oncostatin M (OSM), TNF-β, CD154, lymphotoxin beta (LT-β), an A proliferation-inducing ligand (APRIL), CD70, CD153, glucocorticoid-induced TNF receptor ligand (GITRL), tumor necrosis factor superfamily member 14 (TNFSF14), OX40L (CD252), TALL-1 (Tumor necrosis factor ligand superfamily member 13B—TNFSF13B), TNF-related apoptosis-inducing ligand (TRAIL), TNF-related activation-induced cytokine (TRANCE), erythropoietin (Epo), thyroid peroxidase precursor (Tpo), FMS-related tyrosine kinase 3 ligand (FLT-3L), stem cell factor (SCF), macrophage colony-stimulating factor (M-CSF), merozoite surface protein (MSP), a Nucleotide-binding oligomerization domain-containing protein (NOD) ligand (e.g., NOD1, NOD2, or NOD1/2 agonists), a RIG-I-like receptor (RLR) ligand (e.g., 5′ppp-dsRNA, 3p-hpRNA, Poly(I:C), or Poly(dA:dT)), a C-type lectin receptor (CLR) ligand (e.g., curdlan, β-glucan, HKCA, laminarin, pustulan, scleroglucan, WGP dispersible, WGP soluble, zymosan, zymosan depleted, furfurman, b-GlcCer, GlcC14C18, HKMT, TDB, TDB-HS15, or TDM), a cyclic dinucleotide sensor ligand (e.g., C-Gas agonist or stimulator of interferon gene (STING) ligand), an inflammasome inducer (e.g., alum, ATP, CPPD crystals, hemozoin, MSU crystals, Nano-SiO2, Nigericin, or TDB), an aryl hydrocarbon (AhR) ligand (e.g., FICZ, indirubin, ITE, or L-kynurenine), an alpha-protein kinase 1 (ALPK1) ligand, a multi-PRR ligand, an NFKB/NFAT activator (e.g., concavalin A, ionomycin, PHA-P, or PMA) or a combination thereof. In some embodiments, a cytokine comprises IFN-β.

In some embodiments of the present disclosure, a step of culturing an immune cell (e.g., a monocyte, macrophage, or dendritic cell) occurs after a step of delivering an mRNA to the immune cell.

In some embodiments, culturing a modified immune cell (e.g., a monocyte, macrophage, or dendritic cell) with a cytokine or immune stimulating recombinant protein increases the viability of the modified immune cell relative to a modified immune cell of the same type that was not cultured with the cytokine or immune stimulating recombinant protein. In some embodiments, culturing a modified immune cell (e.g., a monocyte, macrophage, or dendritic cell) with a cytokine or immune stimulating recombinant protein increases expression of an exogenous fibrolytic agent (e.g., a polypeptide described herein) by the modified immune cell relative to a modified immune cell of the same type that was not cultured with the cytokine or immune stimulating recombinant protein. In some embodiments, culturing a modified immune cell (e.g., a monocyte, macrophage, or dendritic cell) with a cytokine or immune stimulating recombinant protein increases expression of an exogenous anti-inflammatory agent (e.g., a polypeptide described herein) by the modified immune cell relative to a modified immune cell of the same type that was not cultured with the cytokine or immune stimulating recombinant protein.

In some embodiments, culturing a modified immune cell (e.g., a monocyte, macrophage, or dendritic cell) with a cytokine or immune stimulating recombinant protein increases longevity of expression of an exogenous fibrolytic agent by the modified immune cell relative to a modified immune cell of the same type that was not cultured with the cytokine or immune stimulating recombinant protein. In some embodiments, culturing a modified immune cell (e.g., a monocyte, macrophage, or dendritic cell) with a cytokine or immune stimulating recombinant protein increases longevity of expression of an exogenous anti-inflammatory agent by the modified immune cell relative to a modified immune cell of the same type that was not cultured with the cytokine or immune stimulating recombinant protein.

In some embodiments, culturing a modified immune cell (e.g., a monocyte, macrophage, or dendritic cell) with a cytokine or immune stimulating recombinant protein increases effector activity of the modified immune cell relative to a modified immune cell of the same type that was not cultured with the cytokine or immune stimulating recombinant protein. In some embodiments, culturing a modified immune cell (e.g., a monocyte, macrophage, or dendritic cell) with a cytokine or immune stimulating recombinant protein increases M2 polarization of the modified immune cell relative to a modified immune cell of the same type that was not cultured with the cytokine or immune stimulating recombinant protein.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### EXAMPLES

The following examples are provided so as to describe to the skilled artisan how to make and use methods and compositions described herein, and are not intended to limit the scope of the present disclosure.

### Example 1: Engineered, Polarized Macrophages Secreting Anti-Fibrotic and/or Anti-Inflammatory Proteins

The aim of this Example is to optimize macrophage differentiation to generate macrophages conducive for fibrolytic agent (e.g., anti-fibrotic protein) and/or anti-inflammatory agent (e.g., anti-inflammatory protein) production. The impact of macrophage differentiation methods on steady-state production of fibrolytic MMPs, Pentraxin-2, IL10, CX3CL1, TWEAK, and other transgenes and the effect of polarization of macrophages (M0, M1, and M2 subtypes) on fibrolytic function will be examined. Table 1 shows exemplary polypeptide sequences that may be used for fibrolytic agents and/or anti-inflammatory agents.

TABLE 1
Polypeptide sequences (UniProt KB codes) for fibrolytic
agents and anti-inflammatory agents described herein.

| Protein | Uniprot KB Code |
|---|---|
| MMP9 | P14780 |
| MMP13 | P45452 |
| CXC3CL1 | P78423 |
| IL10 | P22301 |
| Pentraxin-2 | P02743 |

Human anti-fibrotic and/or anti-inflammatory can be introduced into macrophages via electroporation and/or transfection with DNA, mRNA, or chemically modified mRNA or through viral transduction with a lentiviral, adenoviral or alternative viral vector. A macrophage can be one or both of electroporated and/or transfected with at least one VPX mRNA prior to transfection with a viral vector described herein (e.g., a lentiviral vector or an adenoviral vector, e.g., an Ad2 vector or an Ad5 vector (e.g., Ad5f35 adenoviral vector, e.g., a helper-dependent Ad5F35 adenoviral vector)).

For lentiviral transduction of human macrophages, optimal conditions to achieve high levels of transduction and expression of anti-fibrotic and/or anti-inflammatory cargo in human macrophages will be determined. After optimizing transduction conditions, secretion and kinetics of MMPs, Pentraxin 2, IL10, CX3CL1, or other transgenes will be determined using ELISA or Meso Scale Discovery (MSD). The mRNA levels (mRNA half-life) of MMPs, Pentraxin 2, IL10, and CX3CL1 will be determined using qRT-PCR. Functional activity of secreted factors will also be confirmed.

For mRNA transfection of human macrophages, engineered human macrophages will be generated by applying non-viral methods to evaluate anti-fibrotic and/or anti-inflammatory cargo expression. After optimizing transfection conditions, secretion and kinetics of MMPs, Pentraxin 2, IL10, CX3CL1, or other transgenes will be determined using ELISA or MSD. The mRNA levels (mRNA half-life) of MMP, Pentraxin 2, IL10, CX3CL1, or other transgenes will be determined using qRT-PCR. Functional activity of secreted factors will also be confirmed.

The effect of the fibrotic liver environment on engineered macrophages and their cargo secretion will be determined in vitro. Engineered macrophages will be exposed to TNFα, IL1β, IL6, and IFN [symbol] . For macrophages engineered to express MMP or other enzymes, supernatant will then be collected and analyzed qualitatively for enzyme activity. Secretion and kinetics of MMPs, Pentraxin 2, IL10, CX3CL1, or other transgenes will also be tested qualitatively using ELISA.

A syngeneic murine system for evaluation of mouse macrophage engineering may be established. The stability of anti-fibrotic and anti-inflammatory secreted proteins from engineered mouse macrophages may be determined using ELISA or MSD in vitro. The effect of the fibrotic liver environment on engineered mouse macrophages and their cargo secretion may also be determined in vitro.

### Example 2: Anti-Fibrotic and Anti-Inflammatory Activity of Engineered Macrophages in a Liver Fibrosis Mouse Model

A liver fibrosis model will be developed using NOD.CB17-Prkdc^{scid} mice. Fibrosis will be induced with carbon tetrachloride (CCl₄) via intraperitoneally biweekly injections. Mice will be treated with engineered human macrophages at 8 weeks after fibrosis induction. For Pentraxin-2, mice will be treated with engineered IL-2 expressing human macrophages at 4 weeks after fibrosis induction

Macrophages of different phenotypic polarizations (M0, M1, and M2 subtypes) will be evaluated in this model. Fibrosis will be measured using Pico Sims Red staining for detecting fibrosis of liver sections and pathological scoring. Liver sections will also be examined for fibrosis markers, such as Collagen-Iα deposition, by Immunohistochemistry. Gene expression of pro-fibrotic genes, such as Sma1α and TGFβ, as well as anti-fibrotic liver MMP9 and MMP13 levels will be assessed over time. Immunofluorescence co-staining for macrophage marker F4/80 and MMP9/MMP13/IL10/Pentraxin-2/CX3CL1 will be performed. Hepatic and systemic inflammation will be investigated in addition to examining parameters associated with fibrosis. The liver microenvironment will be evaluated by IHC, FACS, and/or single cell RNA sequencing. The biodistribution and pharmacokinetics of engineered mouse macrophages with anti-fibrotic and anti-inflammatory cargos will be determined.

Functional assessment of engineered mouse macrophages may also be assessed in a syngeneic in vivo liver fibrosis model in immunocompetent mice. Fibrosis will be induced with CCL4 intraperitoneally biweekly injections. Mice will be treated with engineered mouse macrophages at 4-8 weeks after fibrosis induction. Fibrosis will be measured using Pico Sims Red staining for detecting fibrosis of liver sections and pathological scoring. Liver sections will also be examined for fibrosis markers, such as Collagen-I deposition, by Immunohistochemistry. Gene expression of pro-fibrotic genes, such as Sma1α and TGFβ, as well as anti-fibrotic liver MMP9 and MMP13 levels will be assessed. Immunofluorescence co-staining for macrophage marker F4/80 and MMP9/MMP13/IL10/Pentraxin-2/CX3CL1 will be performed. The liver microenvironment and immune infiltration will be evaluated using IHC, FACS, and/or single cell RNA sequencing. Local and systemic inflammatory markers will be measured. The biodistribution and pharmacokinetics of engineered mouse macrophages with anti-fibrotic and anti-inflammatory cargos will be determined.

### Example 3: Anti-Fibrotic Activity of Engineered Macrophages in a NAFLD-NASH Induced Liver Fibrosis Model

The efficacy and anti-fibrotic activity of engineered macrophages will be evaluated in a NAFLD-NASH liver disease model. The efficacy of engineered macrophages will be determined using an American Lifestyle Induced Obesity Syndrome (ALIOS) diet, a Streptozotocin NAFLD model, and a High Fat High Cholesterol Diet. The efficacy of human macrophages in humanized FRG® KO models of NASH and NAFLD will also be determined.

### Example 4: Engineered Macrophages Secreting Anti-Inflammatory or Fibrolytic Agents

The objective of this Example was to engineer macrophages using both non-viral (e.g., mRNA) and viral (e.g., lentiviral) methods of delivery to secrete anti-inflammatory and/or anti-fibrolytic agents. Human macrophages were electroporated with mRNA encoding IL10 or MMP13 mRNA (comprising chemical modifications that enhance viability, expression, and persistence) at different concentrations of mRNA. Production of IL10 and MMP13 was monitored by measuring IL10 or MMP13 levels in supernatant with ELISA at different time points. Human macrophages were transduced with VPX lentivirus (VPX-LV) encoding IL10 or MMP13 at different MOIs. Production of IL10 and MMP13 was monitored by measuring IL10 or MMP13 levels in supernatant with ELISA at different time points.

For the following experiments of human macrophages using electroporation of mRNA encoding IL10, CD14+ monocytes from two donors were differentiated with GM-CSF for 7 Days to convert them into macrophages. Macrophages on Day 0 were electroporated with 30 nM, 100 nM, and 300 nM of IL10 mRNA comprising chemical modifications for stability or mCherry mRNA.

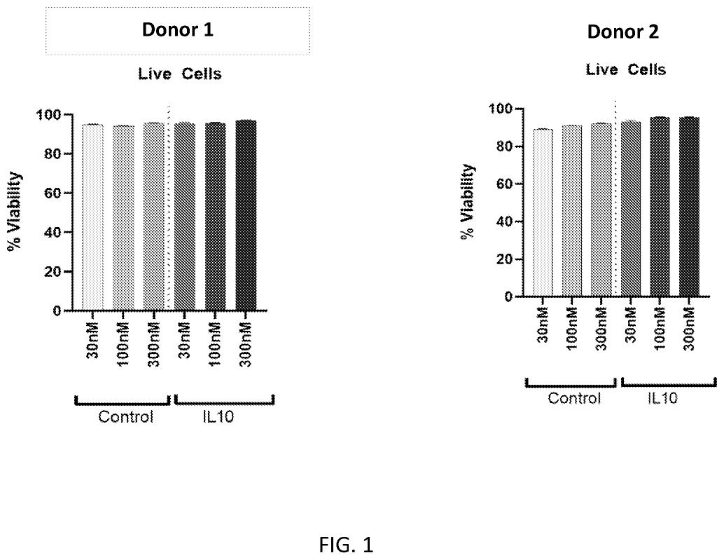

Viability of Macrophages engineered to secrete IL10 was investigated at Day 3. After electroporation, IL10 engineered macrophages were rested and cultured for 3-7 days. Detached cells were stained with Live/Dead Aqua stain. Flow Cytometry analysis was performed to measure viability. Macrophages engineered to secrete IL10 via mRNA electroporation were viable at Day 3 (FIG. 1).

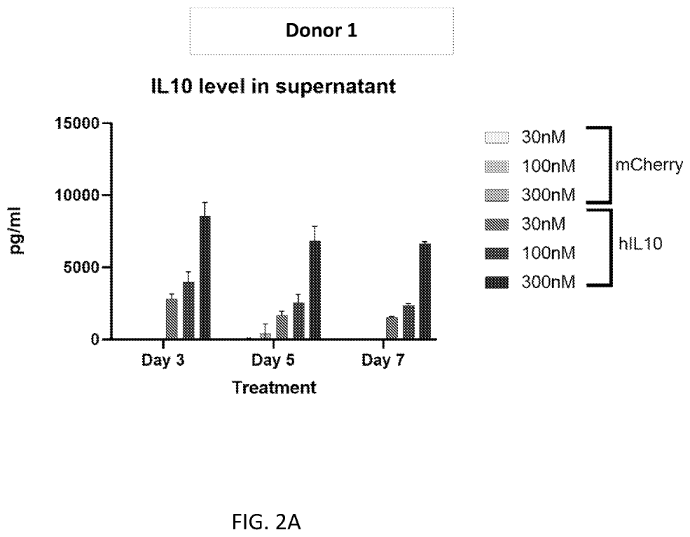

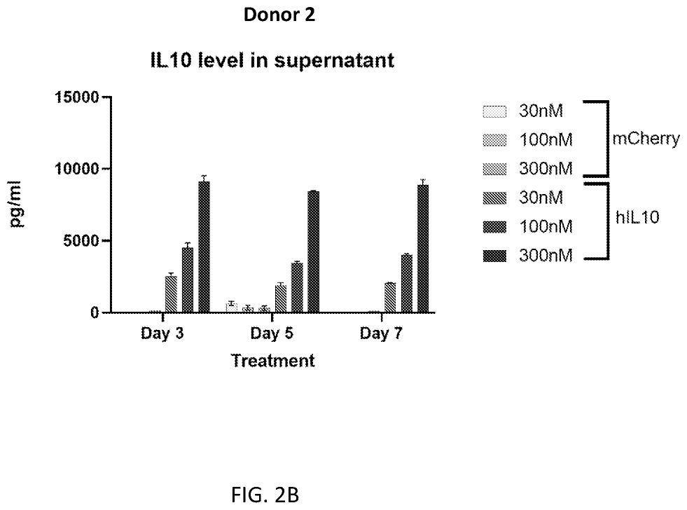

IL-10 secretion of macrophages electroporated with mRNA was investigated. After electroporation, cell culture supernatants were collected at Day 3, Day 5, and Day 7. Supernatant from each time point and each condition was used to run (in duplicates) IL-10 ELISA. Macrophages electroporated with mRNA secreted IL10 at Day 3, Day 5, and Day 7 with increasing IL10 secretion corresponding to increasing mRNA amount (FIGS. 2A-2B).

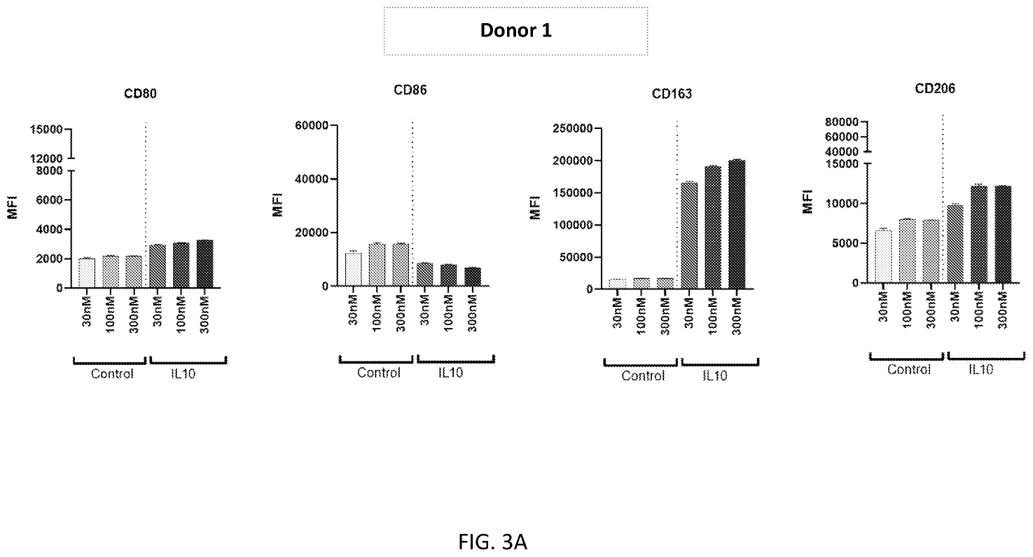

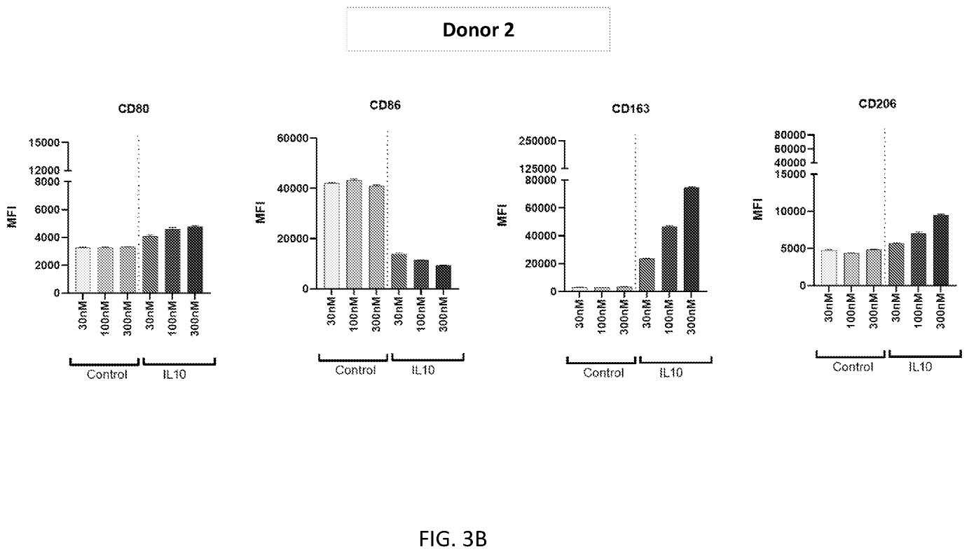

To investigate macrophage polarization on Day 3, IL10 engineered macrophages were detached and evaluated using flow cytometry analysis. Detached cells were stained with Live/Dead Aqua stain, CD163, CD206, CD80, and CD86 antibodies. Flow Cytometry analysis was performed to examine macrophage phenotype. There was a minor increase in the M1 marker CD80, and a minor decrease in the M1 marker CD86 (FIGS. 3A-3B). Overall, the impact of IL10 expression in macrophages on M1 markers was insignificant. In contrast, there was a robust increase in the M2 marker CD163, and a minor increase in the M2 marker CD206. IL10 signals through STAT3 and robustly induces CD163, but not CD206, which is regulated by STAT6. These data demonstrate an “IL-10 M2 phenotype” and prove functional secretion of IL10 as well as the ability of these engineered cells to self-polarize to M2.

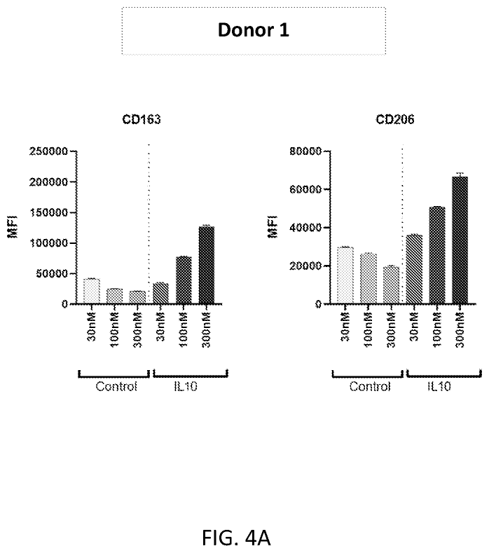

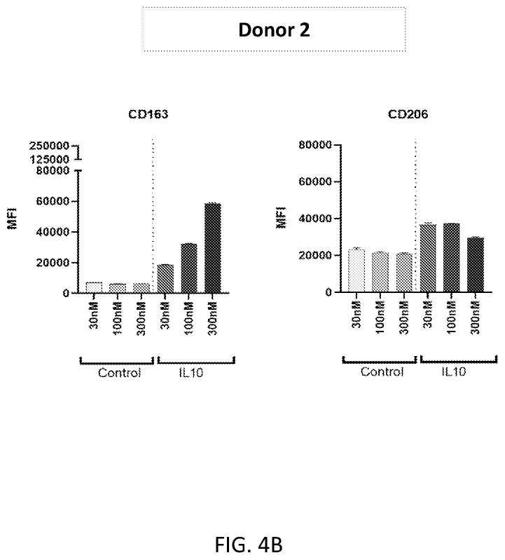

Polarization of IL10 engineered macrophages at Day 7 was investigated. At Day 7, IL10 engineered macrophages plated on Upcell plates detached by keeping the plate on an ice block for 15-20 min. Detached cells were stained with Live/Dead Aqua stain, CD163, and CD206. Flow Cytometry analysis was performed to examine macrophage phenotype. IL10 engineered macrophages maintained M2-like phenotype at Day 7 (FIGS. 4A-4B).

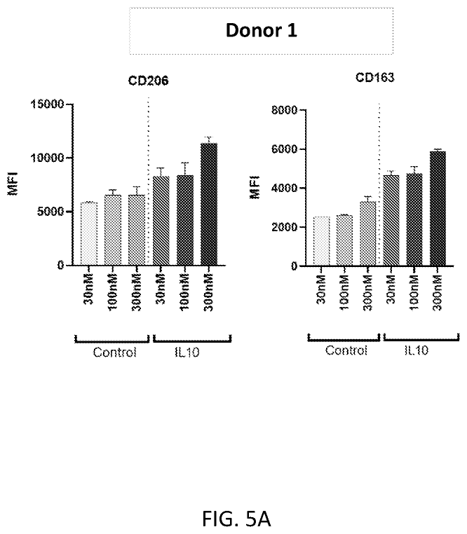

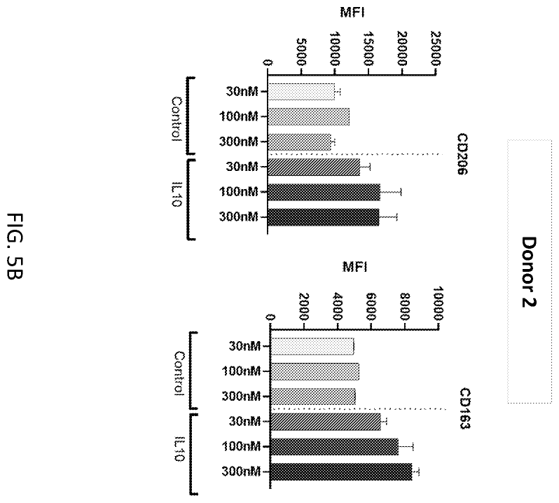

The effect of IL10 secreting macrophages engineered using mRNA on other macrophages was investigated. Supernatants from IL10 engineered macrophages were collected at Day 7 and stored at −80 C. At Day 0, the frozen supernatant was thawed and diluted 1:10 with media. The diluted supernatant was passed through a 0.2 uM filter. The filtrate was used to stimulate 2 new donors. At Day 2 post-stimulation, cells were detached and stained with Live/Dead Aqua stain, CD163, and CD206. Flow Cytometry analysis was performed to examine macrophage phenotype. IL10 secretion from engineered macrophages converted other macrophages into M2-like macrophages (FIGS. 5A-5B). Bystander macrophages upregulated the IL10 response gene CD163. An increase in CD206, another M2 marker, was seen at high IL10 mRNA concentrations.

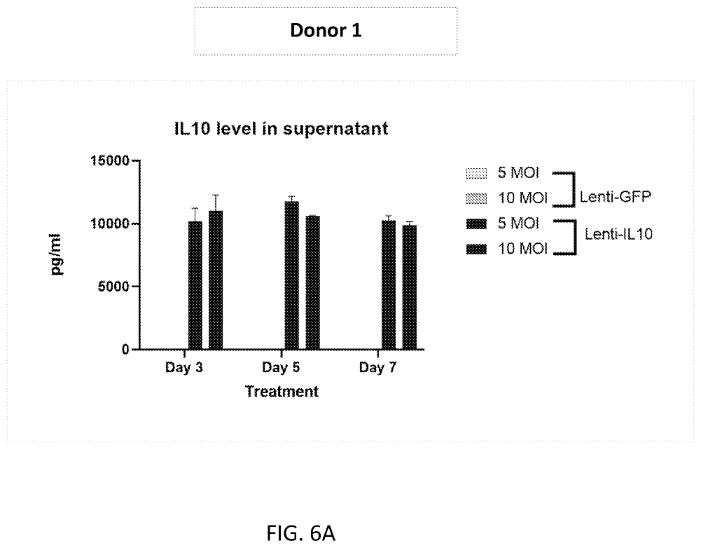

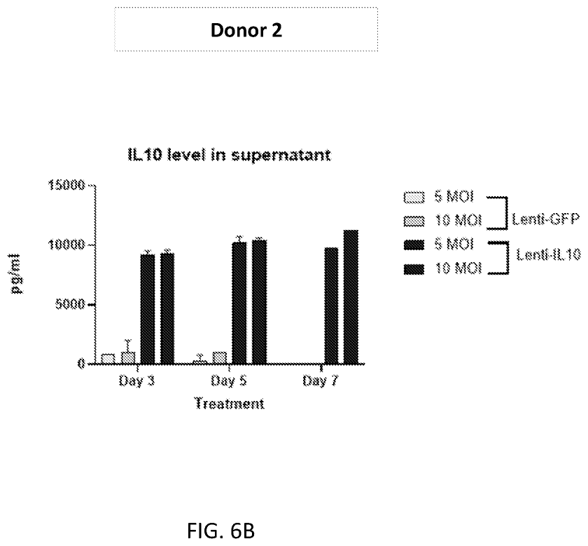

For the following experiments using VPX-LV encoding IL10, CD14+ monocytes were differentiated with GM-CSF for 7 Days to convert them into macrophages. Macrophages on Day 0 were transduced with VPX-LV encoding IL10 or VPX-LV encoding GFP. Post-transduction, cell culture supernatants were collected at Day 3, Day 5, and Day 7. Supernatant from each time point and each condition was used to run (in duplicate) an IL-10 ELISA. Macrophages transduced with VPX-LV secreted IL10 at Day 3, Day 5, and Day 7 (FIGS. 6A-6B).

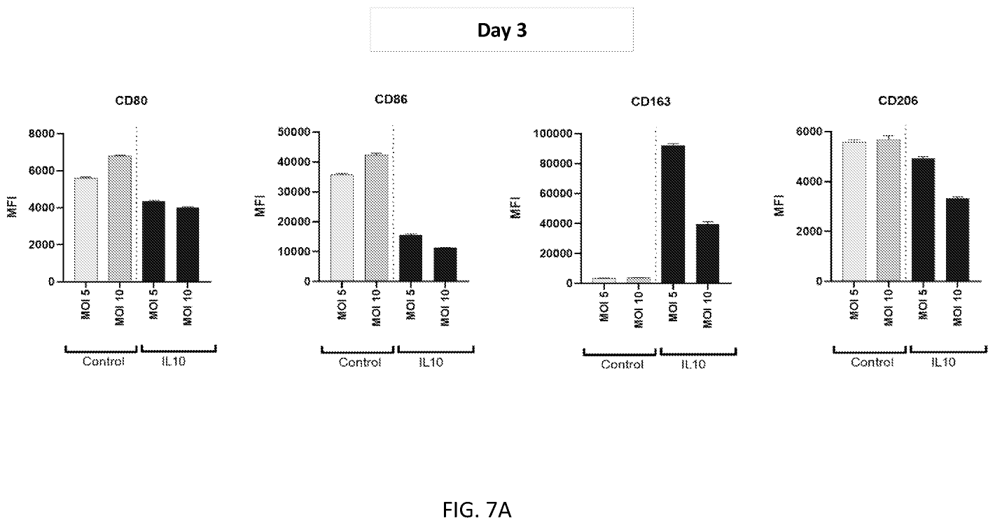

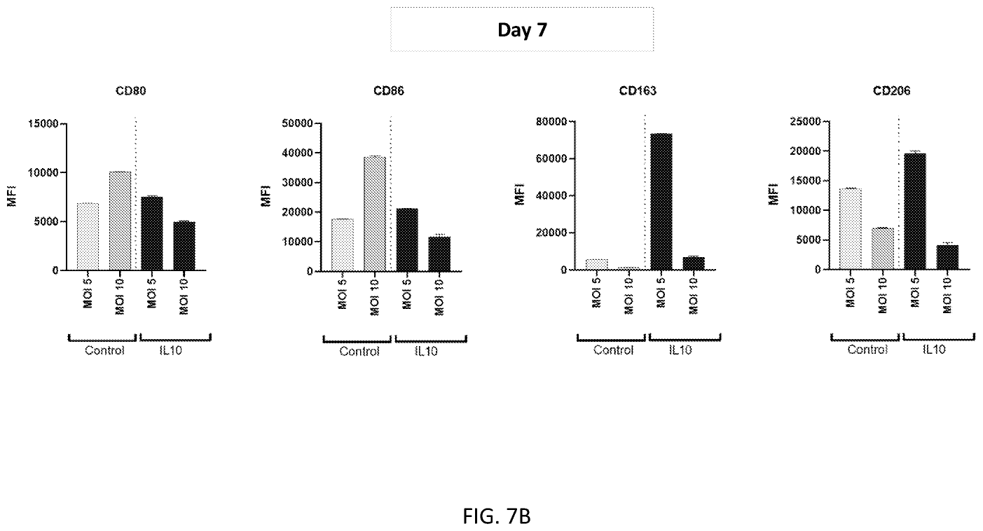

The phenotype of IL10 engineered macrophages was investigated. At Day 3 and Day 7, IL10 engineered macrophages were evaluated using flow cytometry analysis. Detached cells were stained with Live/Dead Aqua stain, CD163, CD206, CD80, and CD86. Flow Cytometry analysis was performed to examine the phenotype of the macrophage. IL10 engineered macrophages converted into an anti-inflammatory M2 phenotype at Day 3 and Day 7 (FIGS. 7A-7B). VPX-LV IL10 engineered human macrophages upregulated the IL10 response M2-marker CD163. Macrophages demonstrated induction of CD163 by day 3. IL10 engineered macrophages maintained an M2-like phenotype at Day 7. Notably, MOI 5 led to significantly more M2 phenotype macrophages than MOI 10, demonstrating that an optimal MOI is required to produce a self-polarizing VPX-LV engineered macrophage secreting IL10.

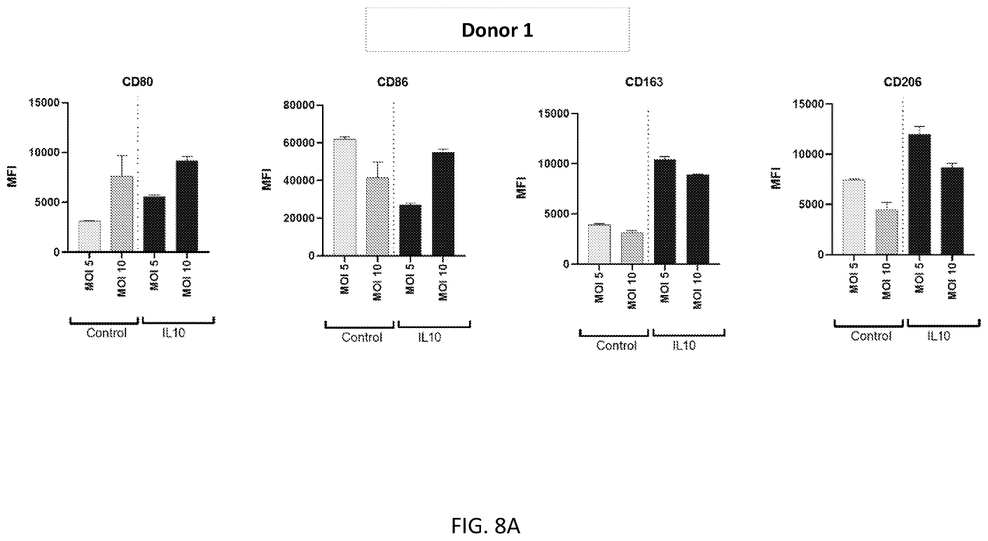

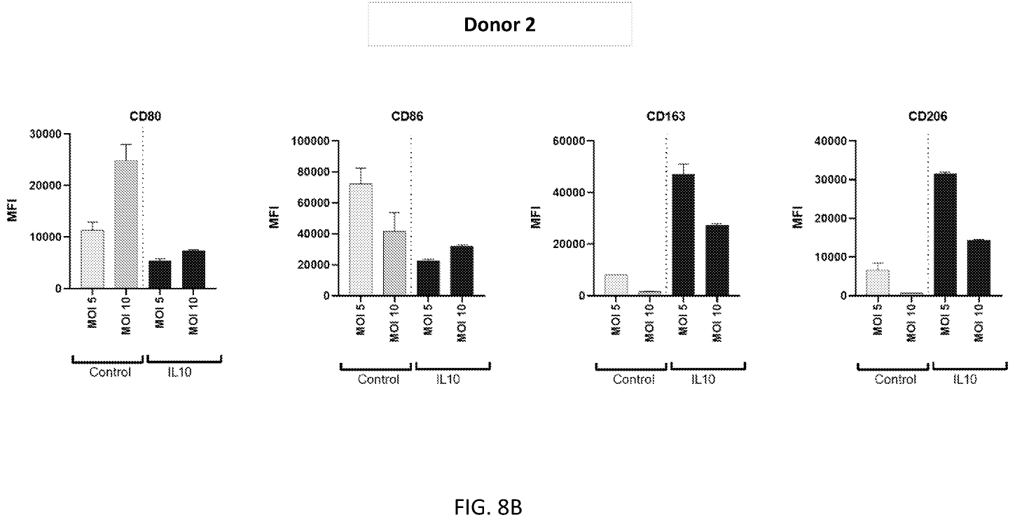

The effect of IL10 secreting macrophages engineered using VPX-LV on other macrophages was investigated. Supernatants from IL10 engineered macrophages were collected at Day 7 and stored at −80 C. At Day 0, the frozen supernatant was thawed and diluted 1:10 with media. The diluted supernatant was passed through a 0.2 uM filter. The filtrate was used to stimulate 2 new donors. At Day 2 post-stimulation, cells were detached and stained with Live/Dead Aqua stain, CD163, CD206, CD80, and CD86. Flow Cytometry analysis was performed to examine macrophage phenotype. IL10 secreted from VPX-LV engineered macrophages converted other macrophages into M2-like macrophages (FIGS. 8A-8B). Bystander macrophages upregulated M2 macrophage markers CD163 and CD206 demonstrating that VPX-LV IL10 engineered macrophages secrete functional IL10 capable of inducing an immunosuppressive phenotype in surrounding immune cells.

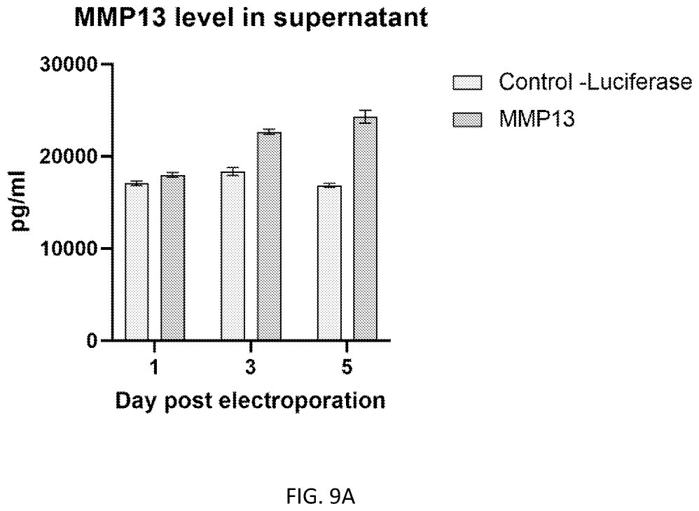

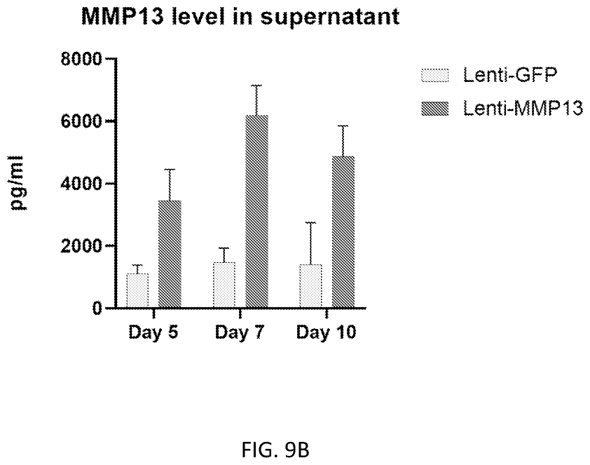

MMP13 secretion from macrophages electroporated with MMP13 mRNA was investigated. CD14+ monocytes were differentiated with GM-CSF for 7 Days to convert them into macrophages. Macrophages on Day 0 were electroporated with 100 nM of MMP13 mRNA comprising chemical modifications or f-luc mRNA. After electroporation, cell culture supernatants were collected at Day 1, Day 3, and Day 5. Supernatant from each time point and each condition was used to run (in duplicate) MMP13 ELISA. Macrophages from the donor evaluated produced MMP13 at baseline. Electroporation with MMP13 mRNA increased the relative amount of MMP13 secreted compared to control macrophages, particularly at day 5 where a >1.5× increase is seen (FIG. 9A). Certain conditions reduce MMP production, and thus, exogenous overexpression ensures therapeutic payload delivery. Further, the donor tested had particularly high background MMP13.

MMP13 secretion from macrophages transduced with MMP13 VPX-LV was investigated. CD14+ monocytes were differentiated with GM-CSF for 7 Days to convert them into macrophages. Macrophages on Day 0 were transduced with VPX-LV encoding MMP13 or VPX-LV encoding GFP. Post-transduction cell culture supernatants were collected at Day 5, Day 7, and Day 10. Supernatant from each time point and each condition was used to run (in duplicates) MMP13 ELISA. There was significantly higher secretion of MMP13 from MMP13 VPX-LV engineered macrophages compared to control VPX-LV GFP macrophages (FIG. 9B).

### CONCLUSIONS

The data provided herein show that human macrophages can be transiently engineered using non-viral mRNA methods to be anti-fibrolytic and anti-inflammatory human macrophages. Engineered cells using mRNA methods were generated with high viability and secreted IL10 or MMP13. Macrophages produced IL10 until at least Day 7. IL10 engineered macrophages self-polarized to an M2 phenotype and maintained M2 phenotype until at least Day 7. Supernatant from IL10 engineered macrophages converted bystander M0 macrophages into M2-like macrophages. Engineered macrophages produced MMP13 starting on Day 3. Maximum secretion of MMP13 was reached by Day 5.

The data provided herein show that human macrophages can be stably engineered using viral methods (VPX-lentivirus transduction) to be anti-fibrolytic and anti-inflammatory human macrophages. Engineered cells using VPX-lentivirus transduction were generated with high viability and secreted IL10 or MMP13. Macrophages produced IL10 starting on Day 3 post-transduction and released IL10 until at least Day 7. IL10 engineered macrophages self-polarized to an M2 phenotype and maintained M2 phenotype until at least Day 7. Supernatant from IL10 engineered macrophages efficiently converted bystander M0 macrophages into M2-like macrophages. Macrophages produced MMP13 starting on Day 5 and maintained secretion until at least Day 10. Maximum secretion of MMP13 was reached by day 7.

These results indicate that primary human macrophages were successfully engineered to secrete IL10 or MMP13 using either non-viral (mRNA comprising chemical modifications) or viral (VPX-lentivirus transduction) methods. Engineered macrophages produced anti-fibrotic (MMP13) or anti-inflammatory (IL10) payloads, which are model payloads in each class.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

1. A modified immune cell comprising one or more nucleic acid sequences encoding:
(i) at least one exogenous fibrolytic agent, and/or
(ii) at least one exogenous anti-inflammatory agent.

2. The modified immune cell of claim 1, wherein the at least one exogenous fibrolytic agent comprises a matrix metallopeptidase (MMP), or TWEAK polypeptide.

3. The modified immune cell of claim 2, wherein the MMP comprises or is one or more of MMP-1, MMP-2, MMP-3, MMP-7, MMP-8, MMP-9, MMP-12, MMP-13, MMP-14, MMP-15, MMP-16, MMP-19, MMP-24, and/or TIMP-1.

4. The modified immune cell of any one of claims 1-3, wherein the at least one exogenous anti-inflammatory agent comprises or is a cytokine, a chemokine, or a pentraxin.

5. The modified immune cell of claim 4, wherein:
(i) the cytokine comprises or is IL10, IL-4, IL-13, and/or TGF-beta;
(ii) the chemokine comprises or is CX3CL; and/or
(iii) the pentraxin comprises or is Pentraxin-2.

6. The modified immune cell of any one of claims 1-5, wherein the at least one exogenous fibrolytic agent and/or the at least one exogenous anti-inflammatory agent are tethered to the immune cell or secreted from the immune cell.

7. The modified immune cell of any one of claims 1-6, wherein the one or more nucleic acid sequences comprise one or more liver specific promoters or cirrhosis specific promoters.

8. The modified immune cell of any one of claims 1-7, wherein the one or more nucleic acid sequences comprise a CX3CR1 promoter, an insulin-like growth factor 1 (IGF1), or a CD11B promoter.

9. The modified immune cell of any one of claims 1-8, wherein the modified immune cell comprises a macrophage, monocyte, or dendritic cell.

10. The modified immune cell of claim 9, wherein the macrophage comprises or is a polarized macrophage.

11. The modified immune cell of claim 10, wherein the polarized macrophage comprises or is an M0, M1, or M2 macrophage.

12. The modified immune cell of claim 11, wherein the M2 macrophage comprises or is a M2A, M2B, M2C, or M2D macrophage.

13. The modified immune cell of any one of claims 9-12, wherein the macrophage is derived from a monocyte or a precursor immune cell.

14. The modified immune cell of claim 13, wherein the precursor immune cell comprises or is a hematopoietic stem cell, myeloid progenitor, myeloblast, monoblast, promonocyte, or an intermediate thereof.

15. The modified immune cell of any one of claims 9-14, wherein the macrophage is a G-MCSF derived macrophage or an M-CSF derived macrophage.

16. A pharmaceutical composition comprising a modified immune cell of any one of claims 1-15.

17. The pharmaceutical composition of claim 16, comprising a pharmaceutically acceptable carrier.

18. A nucleic acid construct comprising one or more nucleic acid sequences encoding least one exogenous fibrolytic agent and/or at least one exogenous anti-inflammatory agent.

19. A pharmaceutical composition comprising the nucleic acid construct of claim 18.

20. The pharmaceutical composition of claim 19, comprising a pharmaceutically acceptable carrier.

21. A method for treating or preventing fibrosis or inflammation in a subject, comprising delivering to the subject a therapeutically effective amount of the pharmaceutical composition of any one of claim 16, 17, 19, or 20.

22. The method of claim 21, wherein the fibrosis comprises or is a liver, lung, heart, vasculature, kidney, pancreas, skin, gastrointestinal, bone marrow, hematopoietic tissue, nervous system, and/or eye fibrotic disease, disorder, or condition.

23. The method of claim 22, wherein the liver fibrotic disease, disorder, or condition comprises a fatty liver disease, disorder, or condition.

24. The method of claim 23, wherein the fatty liver disease, disorder, or condition comprises non-alcoholic fatty liver disease (NAFL) or alcoholic liver disease.

25. The method of claim 24, wherein the NAFL comprises non-alcoholic fatty liver disease (NAFLD) or non-alcoholic steatohepatitis (NASH).

26. The method of claim 24, wherein the alcoholic liver disease comprises alcoholic fatty liver disease (AFLD) or alcoholic steatohepatitis (ASH).

27. The method of any one of claims 21-26, wherein the subject has one or more of cirrhosis, liver damage, hepatocarcinoma, steatosis, an increased risk of liver failure, an increased risk of death, and/or Hepatitis C infection (HCV).

28. The method of any one of claims 21-27, wherein the inflammation comprises or is a liver, gastrointestinal tract, lung, skin, cardiovascular system, nervous system, kidney, pancreas, joint, eye, and/or an endocrine system inflammatory disease, disorder, or condition.

29. The method of any one of claims 21-28, wherein the method reduces activation of hepatic stellate cells.

30. The method of any one of claims 21-29, wherein the method improves liver regeneration and/or liver resolution.

31. The method of any one of claims 21-30, wherein the method balances pro-fibrotic and anti-fibrotic macrophage populations in the subject.

32. A method of modifying an immune cell, comprising delivering to the immune cell a nucleic acid construct comprising one or more nucleic acid sequences encoding at least one exogenous anti-fibrotic agent and/or at least one exogenous anti-inflammatory agent.

33. The method of claim 32, wherein the delivering comprises electroporation or transfection with mRNA, DNA, or chemically modified mRNA.

34. The method of claim 32, wherein the delivering comprises transduction with an adeno-associated viral (AAV) vector, an adenoviral vector, or a retroviral vector.

35. The method of claim 34, wherein the retroviral vector comprises a lentiviral vector or a gammaretroviral vector.

36. The method of claim 35, wherein the lentiviral vector is packaged with a Vpx protein.

37. The method of claim 35, wherein a Vpx protein is delivered to the immune cell either before, concurrently with, or subsequently to the nucleic acid construct.

38. The method of claim 34, wherein the adenoviral vector comprises an Ad2 vector or an Ad5 vector.

39. The method of claim 38, wherein the Ad5 vector comprises an Ad5f35 adenoviral vector.
